(19) United States
(12) Patent Application Publication
(10) Pub. No.: US 2024/0173325 A1
(43) Pub. Date: May 30, 2024

(54) METHODS FOR TREATING SPINAL MUSCULAR ATROPHY

(71) Applicants: Genentech, Inc., South San Francisco, CA (US); Hoffmann-La Roche Inc., Little Falls, NJ (US)

(72) Inventors: Richard S. Finkel, Memphis, TN (US); Heidemarie Kletzl, Basel (CH); Lutz Mueller, Basel (CH); Renata Siciliani Scalco, Basel (CH); Gaurav Seth, Roslyn Heights, NY (US); Alexander Herbert Stephan, Basel (CH)

(73) Assignees: Genentech, Inc., South San Francisco, CA (US); Hoffmann-La Roche Inc., Little Falls, NJ (US)

(21) Appl. No.: 18/380,141

(22) Filed: Oct. 13, 2023

Related U.S. Application Data
(60) Provisional application No. 63/416,416, filed on Oct. 14, 2022.
(60) Provisional application No. 63/428,348, filed on Nov. 28, 2022.
(60) Provisional application No. 63/434,915, filed on Dec. 22, 2022.

(51) Int. Cl. A61K 31/519; A61K 31/7125; A61K 48/00; A61P 21/00; C07K 14/47; C12N 15/86

(52) U.S. Cl. CPC A61K 31/519; A61K 31/7125; A61K 48/005; A61P 21/00; C07K 14/47; C12N 15/86

(57) ABSTRACT

Provided are methods for the treatment of spinal muscular atrophy (SMA) in a fetal subject in need thereof comprising administering an amount of risdiplam to a carrier of the fetal subject. Administering the amount of risdiplam to the carrier of the fetal subject results in transplacental delivery to the fetal subject, resulting in an increase in SMN protein production.

### CROSS-REFERENCE TO RELATED APPLICATIONS SECTION

This application claims the benefit of U.S. Provisional Patent Application Ser. 63/416,416 filed on Oct. 14, 2022, U.S. Provisional Patent Application Ser. 63/428,348 filed on Nov. 28, 2022, and U.S. Provisional Patent Application Ser. 63/434,915 filed on Dec. 22, 2022, the entire contents of which are incorporated herein by reference.

### BACKGROUND

Spinal muscular atrophy (SMA), in its broadest sense, is described as a collection of inherited and acquired central nervous system (CNS) diseases characterized by progressive motor neuron loss in the spinal cord and brainstem causing muscle weakness and muscle atrophy. The most common form of SMA is caused by mutations in the Survival Motor Neuron (SMN) gene and manifests over a wide range of severity affecting infants through adults. See Crawford and Pardo (1996) Neurobiol. Dis., 3:97.

The SMN gene has been mapped by linkage analysis to a complex region in chromosome 5q. In humans, this region contains an approximately 500 thousand base pairs (kb) inverted duplication resulting in two nearly identical copies of the SMN gene. SMA is caused by an inactivating mutation or deletion of the telomeric copy of the gene (SMN1) in both copies (alleles) of chromosome 5, resulting in the loss of SMN1 gene function. However, all patients retain the centromeric copy of the gene (SMN2), and the copy number of the SMN2 gene in SMA patients generally correlates inversely with the disease severity; i.e., patients with less severe SMA have more copies of SMN2. Approximately 73% of individuals with Type 1 SMA have two copies of SMN2 (“SMN2X2”). See Calucho (2018) Neuromuscul. Disord. 28:208-215.

Nevertheless, SMN2 is unable to compensate completely for the loss of SMN1 function due to alternative splicing of exon 7 caused by a translationally silent C to T mutation in exon 7. As a result, the majority of transcripts produced from SMN2 lack exon 7 (Δ7 SMN2), and encode a truncated SMN protein that has an impaired function and is rapidly degraded.

The SMN protein is thought to play a systemic role in RNA processing and metabolism, having a well-characterized function of mediating the assembly of a specific class of RNA-protein complexes termed snRNPs. In particular, SMN protein plays a role in RNA processing and metabolism in motor neurons. Decreased levels of functional SMN protein impact this RNA processing and metabolism, leading to clinical symptoms.

In most cases, SMA is diagnosed based on clinical symptoms and testing for the presence of any functional copies of the SMN1 gene. In some cases, when the SMN1 gene test is not feasible or does not show any abnormality, other tests such as an electromyography (EMG) or muscle biopsy may be indicated.

The clinical spectrum of SMA disorders has been divided into the following five groups.
(a) Type 0 SMA (e.g., In Utero SMA) is the most severe form of the disease and begins before birth. Usually, the first symptom of Type 0 SMA is reduced movement of the fetus in the womb that can first be observed between 30 and 36 weeks of pregnancy. After birth, these newborns have little movement, have difficulties with swallowing and breathing, and often pass away after birth.
(b) Type 1 SMA (Infantile SMA or Werdnig-Hoffmann disease) presents symptoms between 0 and 6 months of SMA and is also very severe. Patients never achieve the ability to sit independently, and death usually occurs within the first 2 years without ventilatory support.
(c) Type 2 SMA (Intermediate SMA) has an age of onset at 7-18 months. Patients achieve the ability to sit unsupported, but never stand or walk unaided. Prognosis in this group is largely dependent on the degree of respiratory involvement.
(d) Type 3 SMA (Juvenile SMA or Kugelberg-Welander disease) is generally diagnosed after 18 months. Type 3 SMA individuals are able to walk independently at some point during their life, but often become wheelchair-bound during youth or adulthood.
(e) Type 4 SMA (Adult onset SMA) results in weakness in late adolescence to adult years in the legs, or in the hands or feet, then progresses to other areas of the body. The course of adult SMA is much slower and has little or no impact on life expectancy.

Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal and all forms have no known cure. The course of SMA is directly related to the rate of motor neuron cell deterioration and the resulting severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum.

Neuropathological studies demonstrate substantial loss of motor neurons and motor neuron pathology in the brainstem and spinal cord up to and including the third trimester of gestation for a fetus with SMA where such loss rapidly increases within the first six months of life. A majority of infants with SMA and two copies of the SMN2 gene demonstrate early symptoms of SMA (including weakness and abnormal reflexes) and score below the tenth percentile on the Hammersmith Neonatal Neurologic Exam test at the first assessment at one to two weeks of age. See Pane (2022) Eur. J. Pediatr. 181(7):2821-2829. Electrophysiological testing of these babies suffering from SMA shows reduced amplitude of the elicited responses on motor nerve conduction study testing, such as ulnar compound muscle action potential (CMAP) amplitude, as compared to age-matched healthy neonates, indicating a loss of motor neurons prior to birth and a reduction in functional motor nerve fiber capacity. See Finkel (2022) Brain. 145(7):2247-2249; Strauss (2022) Nat. Med. 28:1381-1389; Baranello (2021) N. Engl. J. Med. 384(10):915-923; Alves (2021) Mol. Ther. Methods Clin. Dev. 23:524-538; Kolb (2016) Ann. Clin. Transl. Neurol. 3:132-145) and De Vivo (2019) Neuromuscul. Disord. 29(11):842-856. Moreover, neonates with SMA have elevated plasma and cerebrospinal fluid neurofilament levels as compared to healthy newborn infants, indicating an active axonopathy and neurodegenerative process in babies who may appear clinically normal. See Darras (2019) Ann. Clin. Transl. Neurol. 6(5):932-944 and De Vivo (2019) Neuromuscul. Disord. 29:842-856.

These studies demonstrate that infants with SMA and at least one copy of the SMN2 gene have already lost substantial motor neurons before term birth. In fact, developing motor neurons have the greatest need for SMN during the period of the third trimester of fetal development through the first three months of postnatal life. Restoring the SMN protein as early as possible upon development of tissues including motor neurons is thought to result in the greatest benefit to a subject suffering from SMA. See Kong (2021) Sci. Transl. Med. 13(578):eabb6871; Ramos (2019) J. Clin. Invest. 129(11): 4817-4831; Iwatani (2017) Front. Pediatr. 5:194; Martinez-Hemindez (2013) J. Pathol. 229(1):49-61; and Soler-Botija (2002) Brain. 125(Pt 7):1624-34. As such, identification of a neonatal subject with SMA via newborn screening may miss an opportunity for full rescue even if SMA therapy is initiated soon after birth.

Most subjects with SMA with two copies of the SMN2 gene have Type 1 SMA, the most severe form of the disease. See Calucho, et al. (2018) Neuromuscul. Disord. 38:208-15. Even when these patients are diagnosed early through newborn screening, they already have early neurologic signs evident during the first consultation (3-13 days after birth). See Pane, et al. (2022) Eur. J. Pediat. 181:2821-9. The emerging data from studies in presymptomatic patients with SMA treated with current disease-modifying therapies (DMTs) indicates that even subjects who are still asymptomatic at treatment initiation, may not follow a normal developmental trajectory (e.g., will still demonstrate muscle weakness and/or will not be able to stand or walk independently). See De Vivo, et al. (2019) Neuromuscl. Disord. 29:842-56; Strauss, et al. (2022) Nat. Med. 28:1381-9; and Strauss, et al. (2022) Nat. Med. 28:1390-7.

Various treatments for SMA exist, with the goal being to increase SMN levels in the patient. SMN2 splice modification therapy is a treatment option for SMA. Nusinersen (sold under the brand name SPINRAZA®) is an SMN2-directed antisense oligonucleotide that modulates SMN2 splicing to increase SMN protein levels. See Ando (2020) Sci. Rep. 10(1):17472. SPINRAZA® was approved by the U.S. Food and Drug Administration (FDA) in Dec. of 2016 to treat pediatric and adult patients with SMA.

Another SMN2 splice modification therapy, which is the first oral SMA therapy, is risdiplam for the treatment of SMA in pediatric and adult patients. Risdiplam is described chemically as 7-(4,7-diazaspiro[2.5]octan-7-yl)-2-(2,8-dimethylimidazo[1,2- b]pyridazin-6-yl)- 4H-pyrido[1,2-a]pyrimidin-4-one. Risdiplam is commercially available as an oral solution that includes 60 mg of risdiplam as a powder for constitution to provide 0.75 mg/mL solution, sold as Evrysdi®. Evrysdi® is indicated for the treatment of SMA in pediatric and adult patients.

Evrysdi® is administered orally once daily using an oral syringe. Specifically, for a patient less than two months of age, the recommended daily dosage of Evrysdi® is 0.15 mg/kg. For a patient between two months of age and two years of age, the recommended daily dosage of Evrysdi® is 0.2 mg/kg. For a patient two years of age and older weighing less than 20 kg, the recommended daily dosage of Evrysdi® is 0.25 mg/kg. Additionally, for a patient two years of age and older weighing 20 kg or more, the recommended daily dosage of Evrysdi® is 5 mg.

SMN1 expressing gene therapy is another treatment option for SMA. Onasemnogene abeparvovec (also known as “onasemnogene abeparvovec-xioi” or “AVXS-101” and sold under the brand name Zolgensma®) is an adeno-associated virus vector-based gene therapy indicated for the treatment of pediatric patients less than 2 years of age (FDA label for the USA) or under 21 kg weight (EMA approval for the EU) with SMA with bi-allelic mutations in the SMN1 gene. Onasemnogene abeparvovec restores SMN production via one-time systemic administration. See Thomsen (2021) Nat. Med. 27(10):1701-1711; and Hoy (2019) Drugs. 79(11):1255-1262.

Current SMA therapies have improved survival and motor function in symptomatic individuals with Type 1 SMA, especially if treated shortly after birth when some appear pre-symptomatic, See Mercuri (2022) Nat. Rev. Dis. Primers 8(1):52, these treatment options for SMA (e.g., Nusinersen and AVXS-101) are not known to be able to cross the human placenta and, consequently, would not effectively address SMA. Treatment initiation following birth in individuals with SMA having 2 copies of the SMN2 gene who are highly likely to develop Type 1 SMA may only provide a partial response in rescuing vulnerable motor neurons, and the clinical outcome is often sub-optimal. There is, therefore, a need for new therapies for the treatment of SMA to treat the fetus during a critical time when these motor neurons are in particular need of sufficient levels of SMN protein to prevent premature deterioration before birth and to optimize the response to treatment of subjects with SMA.

Specifically, there is a need for new therapies that administer SMA therapy to a subject diagnosed with SMA prior to birth. Thus, in the case of prenatal diagnosis of SMA with, e.g., 2 SMN2 copies, an intervention at the fetal stage with an SMN protein-enhancing agent that crosses the placenta administered orally to the carrier may be of benefit to support the prenatal development of the child and render better postnatal outcomes.

### SUMMARY

Provided herein are methods of treating SMA in a fetal subject in need thereof comprising administering an amount of risdiplam to a carrier of the fetal subject. Administering the amount of risdiplam to the carrier of the fetal subject resulted in transplacental delivery of a therapeutically effective amount of risdiplam to the fetal subject further resulting in an increase in the production of SMN protein across certain tissues, including the central nervous system (CNS) and the spinal motor neurons within. In some embodiments, risdiplam is orally administered to a carrier of the fetal subject. Oral administration may take any suitable form, including solution or tablet.

Various embodiments are contemplated herein. For example, in Embodiment 1, provided is a method of treating Type 0 or Type 1 SMA in a fetal subject in need thereof, the method comprising administering a first amount of risdiplam to a carrier of the fetal subject.

Embodiment 2: The method of Embodiment 1, wherein administering the first amount of risdiplam to the carrier of the fetal subject results in administration of a therapeutically effective amount to the fetal subject.

Embodiment 3: The method of any of Embodiments 1 to 2, wherein the fetal subject is in its third trimester of development.

Embodiment 4: The method of Embodiment 3, wherein the fetal subject is at about twenty-eight weeks to about thirty-eight weeks gestation.

Embodiment 5: The method of any of Embodiments 1 to 4, wherein the fetal subject has no detectable functional survival motor neuron 1 (SMN1) gene.

Embodiment 6: The method of any of Embodiments 1 to 5, wherein the fetal subject has at least one survival motor neuron 2 (SMN2) gene.

Embodiment 7: The method of any of Embodiments 1 to 5, wherein the fetal subject has two or less survival motor neuron 2 (SMN2) gene copies.

Embodiment 8: The method of any of Embodiments 1 to 7, wherein administering the first amount of risdiplam to the carrier of the fetal subject occurs orally.

Embodiment 9: The method of any of Embodiments 1 to 8, wherein administering the first amount of risdiplam to the carrier of the fetal subject occurs daily.

Embodiment 10: The method of any of Embodiments 1 to 9, wherein the first amount of risdiplam is administered at a dose of about 5 mg.

Embodiment 11: The method of Embodiment 1, further comprising terminating administration of the first amount of risdiplam to the carrier of the fetal subject upon delivery of the fetal subject from the carrier as a neonatal subject.

Embodiment 12: The method of Embodiment 11, wherein the neonatal subject has an elevated neurofilament level in at least cord blood.

Embodiment 13: The method of any of Embodiments 11 to 12, further comprising administering a second SMA therapy to the neonatal subject.

Embodiment 14: The method of Embodiment 13, wherein the initiation of administration to the neonatal subject of the second SMA therapy occurs within a time period from delivery of the neonatal subject to about sixty days following delivery of the neonatal subject.

Embodiment 15: The method of any of Embodiments 13 to 14, wherein the second SMA therapy is an SMN expressing gene therapy or an SMN2 splice modification therapy.

Embodiment 16: The method of Embodiment 15, wherein the second SMA therapy is an SMN2 splice modification therapy comprising administering risdiplam to the neonatal subject.

Embodiment 17: The method of Embodiment 16, wherein the amount of risdiplam administered to the neonatal subject is an amount equal to or less than 0.15 mg/kg.

Embodiment 18: The method of any of Embodiments 16 to 17, wherein risdiplam is administered to the neonatal subject if the level of risdiplam in blood of the neonatal subject is less than about 100 ng/mL.

Embodiment 19: The method of Embodiment 18, wherein the level of risdiplam in blood of the neonatal subject is in cord blood of the neonatal subject at the time of delivery.

Embodiment 20: The method of Embodiment 19, wherein the mean area under a concentration-time curve at steady state (AUCₛₛ)level of the risdiplam in the cord blood of the neonatal subject at the time of delivery corresponds to a mean area under a concentration-time curve at steady state (AUCₛₛ) of is less than about 2000 ng·h/mL.

Embodiment 21: The method of any of Embodiments 14 to 20, wherein administering the second amount of risdiplam to the neonatal subject occurs daily.

Embodiment 22: The method of any of Embodiments 14 to 21, wherein administering the second amount of risdiplam to the neonatal subject occurs orally.

Embodiment 23: The method of any of Embodiments 1 to 22, wherein risdiplam is administered as a pharmaceutical composition further comprising ascorbic acid, disodium edetate dihydrate, isomalt, mannitol, polyethylene glycol 6000, sodium benzoate, strawberry flavor, sucralose, and tartaric acid.

Embodiment 24: A method of treating spinal muscular atrophy (SMA) in a subject in need thereof, the method comprising administering to the subject a therapeutically effective amount of an SMA therapy, wherein the subject received risdiplam in utero through a carrier of the subject as a fetal subject. In some such embodiments, the subject is an infant from birth or older. In some such embodiments, the subject is a neonatal subject. In embodiments, the subject suffers from Type 0, Type 1, Type 2, Type 3 or Type 4 SMA. In embodiments, the SMA therapy is an SMN expressing gene therapy or an SMN2 splice modification therapy.

Embodiment 25: A method of treating spinal muscular atrophy (SMA) in a subject in need thereof, the method comprising: administering to a carrier of a fetal subject a first amount of risdiplam in a carrier dosing cycle to administer to the fetal subject a therapeutically effective amount of risdiplam; terminating the carrier dosing cycle when the fetal subject is delivered from the carrier as a neonatal subject; and administrating to the neonatal subject a second amount of risdiplam in a neonatal dosing cycle. In embodiments, the neonatal subject is administered the second amount of risdiplam in a neonatal dosing cycle if the level of risdiplam in blood of the neonatal subject is less than about 100 ng/mL.

Embodiment 26: Risdiplam for use in treating SMA in a fetal subject in need thereof.

Embodiment 27: Risdiplam of Embodiment 26, wherein the fetal subject is in its third trimester of development.

Embodiment 28: Risdiplam of any of Embodiments 26 to 27, wherein the fetal subject is at about twenty-eight weeks to about thirty-eight weeks gestation.

Embodiment 29: Risdiplam of any of Embodiments 26 to 27, wherein the fetal subject is at least about thirty-two weeks gestation.

Embodiment 30: Risdiplam of any of Embodiments 26 to 29, wherein the fetal subject has no detectable functional survival motor neuron 1 (SMN1) genes.

Embodiment 31: Risdiplam of any of Embodiments 26 to 30, wherein the fetal subject has at least one survival motor neuron 2 (SMN2) gene.

Embodiment 32: Risdiplam of any of Embodiments 26 to 30, wherein the fetal subject has two or less survival motor neuron 2 (SMN2) gene copies.

Embodiment 33: Risdiplam of any of Embodiments 26 to 32, wherein the risdiplam is administered to the carrier of the fetal subject.

Embodiment 34: Risdiplam of Embodiment 33, wherein administration of the risdiplam to the carrier of the fetal subject occurs orally.

Embodiment 35: Risdiplam of any of Embodiments 33 to 34, wherein administration of the risdiplam to the carrier of the fetal subject occurs daily.

Embodiment 36: Risdiplam of any of Embodiments 33 to 35, wherein the risdiplam is administered to the carrier of the fetal subject at a dose of about 5 mg.

Embodiment 37: Risdiplam of any of Embodiments 33 to 36, wherein administration of the risdiplam to the carrier of the fetal subject is terminated when the fetal subject is delivered as a neonatal subject.

Embodiment 38: Risdiplam of Embodiment 37, wherein the neonatal subject has an elevated neurofilament level in at least cord blood.

Embodiment 39: Risdiplam of any of Embodiments 33 to 38, further comprising administering to the neonatal subject a second SMA therapy.

Embodiment 40: Risdiplam of Embodiment 39, wherein the second SMA therapy is administered to the neonatal subject during a time period from delivery of the fetal subject as the neonatal subject to about sixty days following the delivery.

Embodiment 41: Risdiplam of any of Embodiments 39 to 40, wherein the second SMA therapy is an SMN expressing gene therapy or an SMN2 splice modification therapy.

Embodiment 42: Risdiplam of any of Embodiments 39 to 41, wherein the second SMA therapy is an SMN2 splice modification therapy comprising risdiplam.

Embodiment 43: Risdiplam of Embodiment 42, wherein a first administration to the neonatal subject of the SMN2 splice modification therapy comprising risdiplam occurs daily via oral administration at an amount equal to or less than 0.15 mg/kg.

Embodiment 44: Risdiplam of Embodiment 43, wherein the first administration of risdiplam to the neonatal subject occurs if the level of risdiplam in blood of the neonatal subject is less than about 100 ng/mL.

Embodiment 45: Risdiplam of Embodiment 44, wherein the level of risdiplam in blood of the neonatal subject is in the cord blood of the neonatal subject at the time of delivery of the fetal subject as the neonatal subject.

Embodiment 46: Risdiplam of any of Embodiments 41 to 45, wherein a second administration to the neonatal subject of the SMN2 splice modification therapy comprising risdiplam occurs daily via oral administration at an amount equal to or less than 0.20 mg/kg, and wherein the second administration of the risdiplam occurs during a time period of about 2 months to about 2 years after delivery of the neonatal subject.

Embodiment 47: Risdiplam of any of Embodiments 26 to 46, wherein the risdiplam is administered as a pharmaceutical composition further comprising ascorbic acid, disodium edetate dihydrate, isomalt, mannitol, polyethylene glycol 6000, sodium benzoate, strawberry flavor, sucralose, and tartaric acid.

Embodiment 48: Risdiplam of any of Embodiments 26 to 47, wherein the SMA is Type 0 SMA or Type 1 SMA.

### BRIEF DESCRIPTION OF THE DRAWINGS

FIG. 1 includes charts associated with administering an amount of risdiplam to a carrier of the fetal subject and monitoring of the carrier and the fetal subject, according to at least some embodiments disclosed herein.

FIG. 2 is a graph comparing the SMN protein level in whole blood in Example 1 to a reference study, according to at least some embodiments disclosed herein.

FIG. 3 is a graph depicting a mean neurofilament level in blood for a carrier (e.g., carrier) on various dates, in accordance with at least some embodiments disclosed herein.

FIG. 4 is a graph depicting a mean neurofilament level in blood for a neonatal subject (e.g., infant) on various dates, in accordance with at least some embodiments disclosed herein.

FIG. 5 is a graph of a reference study measuring plasma phosphorylated neurofilament heavy subunit (pNF-H) concentration (pg/mL) levels at a baseline in infants <1 year of age without SMA, in accordance with at least some embodiments disclosed herein.

FIG. 6 is a schematic diagram of a study schema associated with Example 2, in accordance with at least some embodiments disclosed herein.

### DETAILED DESCRIPTION

Unless specifically defined otherwise, all technical and scientific terms used herein shall be taken to have the same meaning as commonly understood by one of ordinary skill in the art.

### Definitions

“Treatment”, “treating” or similar phrases refer to obtaining beneficial or desired results, such as clinical results, for a subject, suffering from SMA determined, for example, genetically. Beneficial or desired results include any one or more of alleviating one or more symptoms of the SMA, diminishing the extent of the SMA, delaying or slowing the disease progression, improving quality of life, and ameliorating the disease state.

In some embodiments, treatment results in the subject having less severe symptoms than would be expected based on their genetic analysis and/or the amount of SMN1 protein circulating during pre-treatment.

The term “treating spinal muscular atrophy (SMA)” or “treatment of spinal muscular atrophy (SMA)” includes one or more of the following effects: (i) reduction or amelioration of the severity of SMA; (ii) delay of the onset of SMA; (iii) inhibition of the progression of SMA; (iv) reduction of hospitalization of a subject; (v) reduction of hospitalization length for a subject; (vi) increase of the survival of a subject; (vii) improvement of the quality of life of a subject; (viii) reduction of the number of symptoms associated with SMA; (ix) reduction of or amelioration of the severity of one or more symptoms associated with SMA; (x) reduction of the duration of a symptom associated with SMA; (xi) prevention of the recurrence of a symptom associated with SMA; (xii) inhibition of the development or onset of a symptom of SMA; and/or (xiii) inhibition of the progression of a symptom associated with SMA. More particular, “treating SMA” denotes one or more of the following beneficial effects: (i) a reduction in the loss of muscle strength; (ii) an increase in muscle strength; (iii) a reduction in muscle atrophy; (iv) a reduction in the loss of motor function; (v) an increase in motor neurons; (vii) a reduction in the loss of motor neurons; (viii) protection of SMN deficient motor neurons from degeneration; (ix) an increase in motor function; (x) a maintenance of bulbar function; and/or (xi) a reduction in the loss of pulmonary function. “Treating SMA” may further result in the functional ability, or aid in retaining the functional ability, of a human infant or a human toddler (e.g., a neonatal subject) to perform certain physical activities such as to sit up unaided or for a human infant, a human toddler, a human child or a human adult to stand up unaided, to walk unaided, to run unaided, to breathe unaided, to cough unassisted, to turn during sleep unaided, or to swallow unassisted.

“Survival motor neuron” or “SMN” refers to a protein that is encoded by the SMN1 and the SMN2 genes in humans.

“Survival motor neuron 1” or “SMN1” refers to a telomeric copy of the gene encoding the SMN protein.

“Survival motor neuron 2” or “SMN2” refers to a centromeric copy of the gene encoding the SMN protein. Mutations in the SMN2 gene alone do not lead to SMA. Mutations in both the SMN1 and the SMN2 genes result in embryonic death.

“Treatment cycle” refers to a period over which a set of doses of an SMA therapy is administered to a subject such as to a carrier of a fetal subject or to a neonatal subject.

“Carrier” refers to a pregnant human subject carrying a fetal subject.

“Fetal subject” refers to a human subject prior to being delivered from a carrier. In particular, the “fetal subject” has been diagnosed with SMA prior to being delivered from the carrier. The fetal subject may be referred to herein as human subject.

“Neonatal subject” refers to a human subject once it is delivered from a carrier. A neonatal subject is the delivered fetal subject. For purposes of this application, “neonatal subject” refers to a human subject ages through, for example, 30-60 days and may also refer to ages through infancy up to 2 years of age. The neonatal subject may be referred to herein as human subject.

“Gestation” refers to a period of fetal development inside a carrier.

“First trimester” of development refers to the gestation of a fetal subject between conception and about 13 weeks.

“Second trimester” of development refers to the gestation of a fetal subject between about 14 weeks and about 27 weeks.

“Third trimester” of development refers to the gestation of a fetal subject between about 28 weeks until delivery at about 42 weeks.

In some embodiments, “third trimester” of development refers to the gestation of a fetal subject between about 28 weeks until delivery, which may occur prior to about 42 weeks or after about 42 weeks. In some embodiments, “third trimester” may also refer to the gestation of a fetal subject from about 28 weeks until delivery.

“Delivery” and “Birth” are used interchangeably herein to refer to the removal of the fetal subject from the carrier as a neonatal subject.

“Risdiplam” is an SMN2-directed RNA splicing modifier used as a medicament for the treatment of SMA. Risdiplam is described chemically as 7-(4,7-diazaspiro[2.5]octan-7-yl)-2- (2,8-dimethylimidazo[1,2- b]pyridazin-6-yl)-4H-pyrido[1,2-a]pyrimidin-4-one, having the structural formula:

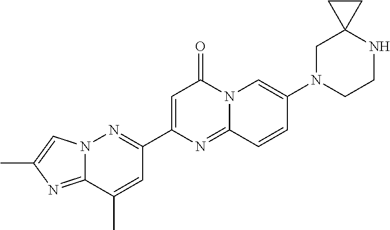 [SMILES: Cc1cn2nc(-c3cc(=O)n4cc(N5CCNC6(CC6)C5)ccc4n3)cc(C)c2n1]

Risdiplam is disclosed in U.S. Published Patent Application No. 2017/1979901 A1 and International Publication No. WO 201/5173181 titled “Compounds for treating spinal muscular atrophy.” Certain crystalline forms of risdiplam are disclosed in U.S. Published Patent Application No. 2021/403487 A1 and International Publication No. WO 2020/079203 titled “New forms of pyrido[1,2-a]pyrimidin-4-one derivatives, its formulation and its process of making.” Risdiplam is primarily metabolized by flavin monooxygenase 1 and 3 (FMO1 and FMO3) and also by CYPs 1A1, 2J2, 3A4, and 3A7. The metabolites of the risdiplam are thought to be inactive. It is thought that risdiplam crosses the placenta in rodents and rabbits and does not interfere with other placental transport mechanisms at a dose that yields embryonal exposure proportionate to the approved dose of 5 mg daily for humans greater than 20 kg (e.g., pregnant carriers). In pre-clinical studies with dosing of pregnant animals, the risdiplam dose was increased in the rats and the rabbits until clear maternal toxicity was observed to establish the NOAEL. In rat fetuses, with dams treated at about 1 mg/kg/day to about 7.5 mg/kg/day, weight loss and minor differences in skeletal ossification were noted. Some rabbit fetuses with dams treated at about 1 mg/kg/day to about 12 mg/kg/day had reduced weight gain and cranium or brain variations, and at about 12 mg/kg/day, some developed hydrocephalus, an abnormally small gall bladder, and lung variations. Humans up to about 20 kg were treated with 0.15 mg/kg/day to 0.25 mg/kg/day, with a dose of 5 mg/day for children and adults greater than 20 kg.

The term “mg/kg” refers to a dose amount of, e.g., risdiplam, in milligrams being administered per kilogram of body weight of the subject to be treated. For example, 0.15 mg/kg risdiplam means a dose amount of 0.15 milligram of risdiplam per kilogram of body weight of the neonatal subject to be treated.

“Adverse event” refers to any undesirable clinical occurrence in the fetal subject (as compared to the fetal subject's baseline health) or the neonatal subject (as compared to the neonatal subject's baseline health) and is any untoward medical occurrence defined as an unintended disease or injury or untoward clinical signs (including abnormal laboratory findings) in the fetal subject or the neonatal subject. More specifically, grades of adverse events include those published under the “Common Terminology Criteria for Adverse Events” (or “CTCAE”) published by the U.S. National Cancer Institute (version 4.03 published 14 Jun. 2010). These include mild (grade 1) adverse events which present as mild symptoms not requiring medical intervention; moderate (grade 2) adverse events, which require minimal, local or noninvasive intervention; severe or medically significant (grade 3) adverse events, which are not immediately life-threatening, but may require hospitalization or prolongation of hospitalization; life- threatening (grade 4) adverse events requiring urgent intervention; and adverse event-related death (grade 5).

A “no observed adverse event level” or “NOAEL” denotes a level of exposure of an organism found by experimentation or observation at which there is no biologically or statistically significant increase in a frequency or a severity of any adverse effects of a tested protocol.

Reference to “about” a value or parameter herein includes (and describes) variations that are directed to that value or parameter per se. In embodiments, the term “about” refers to +/−10%, +/−5%, or +/−1%, of the designated value.

The singular forms “a,” “or,” and “the” include plural referents unless the context clearly dictates otherwise.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

### Methods

The present disclosure describes methods of treating SMA, in particular Type 0 and Type 1 SMA, as well as treatment methods for neonates and infants having received treatment for SMA while in utero, thereby addressing an unmet need for this important patient population.

Importantly, the methods provided herein offer a treatment option during a crucial developmental period, which may provide long term and lasting benefits to the subjects.

If there is a risk that the carrier could have a child with SMA, the carrier may undergo a chorionic villus sampling (CVS) test where a sample of cells taken from the placenta are tested, usually during weeks 11 to 14 of pregnancy, and/or the carrier may undergo an amniocentesis during weeks 15 to 20 of pregnancy to determine the number of copies of the SMN gene mutation the fetal subject has inherited. The parents of the fetal subject may also undergo genetic testing, such as blood test, to detect the most common mutation of SMN. After the fetal subject is born as the neonatal subject, the neonatal subject may undergo a genetic blood test to confirm the SMA condition. Other tests may also be performed, such as a physical examination, electromyography/neurophysiology, and/or a muscle biopsy. Electrophysical testing of the neonatal subject may also serve as a prognostic biomarker.

In embodiments, the fetal subject diagnosed by genetic testing as having SMA, e.g., is homozygous deletion or heterozygosity predictive of loss of function of the SMN1 gene and one or two copies of the SMN2 gene. In embodiments, the fetal subject has no detectable functional copies of the SMN1 gene and at least one copy of the SMN2 gene. In embodiments, the fetal subject diagnosed by genetic testing as having SMA has no detectable functional copies of the SMN1 gene and three or less copies of the SMN2 gene. In some embodiments, the fetal subject has at least one SMN2 gene.

Described herein are methods of treating SMA in a fetal subject in need thereof, where the methods include a carrier treatment cycle. In embodiments, the carrier treatment cycle comprises administering to a carrier of the fetal subject a first SMA therapy comprising an SMN2 splice modification therapy. For example, in embodiments, provided is a method of treating Type 0 or Type 1 SMA in a fetal subject in need thereof, where the method includes administering a first amount of risdiplam to the fetal subject through a carrier of the fetal subject during the carrier treatment cycle. In embodiments, administering the first amount of risdiplam to the carrier of the fetal subject results in the administration of a therapeutically effective amount of risdiplam to the fetal subject.

In embodiments, the fetal subject is diagnosed as having SMA by genetic testing prior to initiation of the carrier treatment cycle. In embodiments, the carrier treatment cycle is initiated when fundamental embryogenesis in the fetal subject is substantially complete. In embodiments, the carrier treatment cycle is initiated when the fetal subject is in its third trimester of development. In embodiments, the carrier treatment cycle is initiated when the gestation of the fetal subject is at about twenty-eight weeks to about thirty-eight weeks gestation. In embodiments, the carrier treatment cycle is initiated when the gestation of the fetal subject is at least about thirty-two weeks gestation. In embodiments, the carrier treatment cycle is initiated when the gestation of the fetal subject is at least about twenty-eight weeks. In embodiments, the carrier treatment cycle is initiated when the gestation of the fetal subject is at about twenty-eight weeks to about thirty weeks gestation.

In embodiments, administering the first amount of risdiplam to the fetal subject comprises oral administration to the carrier of the fetal subject. In embodiments, the first amount of risdiplam is administered daily to the carrier of the fetal subject. In embodiments, administering the first amount of risdiplam to the carrier of the fetal subject comprises daily oral administration of risdiplam to the carrier of the fetal subject. In embodiments, the first amount of risdiplam is a dose of about 5 mg.

In embodiments, the methods further include terminating the carrier treatment cycle when an event occurs. In embodiments, the event includes the delivery of the fetal subject from the carrier. In other embodiments, the carrier treatment cycle is terminated if an adverse change in a biophysical profile of the fetal subject is detected and/or if a non-manageable medical problem arises with respect to the carrier. In embodiments, a baseline pre-treatment full anatomical ultrasound study and/or growth scan of the fetal subject may be performed and may be repeated during the pregnancy of the carrier. The anatomical ultrasound study monitors for fetal growth and any signs of organ maldevelopment, especially in the brain, heart, kidney, and liver. If any adverse findings are identified, the findings are assessed for a possible relationship to risdiplam, and, if deemed to be possibly drug related, then the administration of the first amount of risdiplam to the carrier may be discontinued or terminated. In embodiments, the carrier treatment cycle continues upon delivery of the fetal subject from the carrier enabling the neonatal subject to continue receiving risdiplam through breast milk of the carrier.

In embodiments, the methods further include a neonatal treatment cycle. In embodiments, during the carrier treatment cycle or following termination of the carrier treatment cycle, the neonatal treatment cycle may or may not be initiated. In embodiments, samples of neonatal subject blood (venous and/or arterial) including umbilical cord blood and amniotic fluid are collected at the time of delivery to assess the in utero exposure of the neonatal subject to risdiplam. In embodiments, initiation of the neonatal treatment cycle occurs within a time period from delivery of the neonatal subject. In embodiments, the time period is in a range of from birth to about sixty days or two months. In embodiments, initiation of the neonatal treatment cycle occurs within thirty days from delivery of the neonatal subject. In embodiments, initiation of the neonatal treatment cycle occurs within 7-10 days from delivery of the neonatal subject. In embodiments, initiation of the neonatal treatment cycle occurs within two days from delivery of the neonatal subject. In embodiments, initiation of the neonatal treatment cycle occurs within twenty-four hours of delivery of the neonatal subject.

In embodiments, the SMN protein level is measured in the neonatal subject during a first instance and during a second instance. In embodiments, the first instance is prior to beginning the neonatal dosing cycle and the second instance is after beginning the neonatal dosing cycle. In some embodiments, the SMN protein level in the neonatal subject increases by at least about 20%, by at least about 30%, by at least about 40%, or by at least about 50%, or between about 20% to about 50%, or between about 30% to about 40%, when comparing the second instance to the first instance. In some embodiments, a time period between the first instance and the second instance may be a few days, a few weeks, two weeks, a month, or two months. In embodiments, the SMN protein level in the neonatal subject increase about 30-40% within a month of being administered the second amount of risdiplam.

In embodiments, the neonatal treatment cycle comprises administering a second SMA therapy directly to the neonatal subject and not through a carrier. In embodiments, the second SMA therapy is administered to the neonatal subject if a level of the risdiplam in blood of the neonatal subject is less than about 100 ng/mL. In embodiments, the second SMA therapy is administered to the neonatal subject within a time period of delivery of the neonatal subject (e.g., within about 30 days after birth) if the risdiplam level in cord blood of the neonatal subject at about the time of delivery is less than about 100 ng/mL.

In embodiments, the second SMA therapy is an SMN expressing gene therapy or an SMN2 splice modification therapy. In embodiments, the neonatal treatment cycle comprises administering an SMN2 splice modification therapy as the second SMA therapy. The SMN2 splice modification therapy may be administered to the neonatal subject within a time period from/after delivery of the neonatal subject. In embodiments, the time period is in a range from birth to about sixty days. In embodiments, initiation of administration to the neonatal subject of the SMN2 splice modification therapy occurs within thirty days from delivery of the neonatal subject. In embodiments, initiation of administration to the neonatal subject of the SMN2 splice modification therapy occurs within 7-10 days from delivery of the neonatal subject. In embodiments, initiation of administration to the neonatal subject of the SMN2 splice modification therapy occurs within 1-2 days from delivery of the neonatal subject.

In embodiments, the SMN2 splice modification therapy comprises administering to the neonatal subject, a second amount of risdiplam (the first amount being the amount of risdiplam administered to the subject as a fetal subject through the carrier). In embodiments, the second amount of risdiplam is administered to the neonatal subject if the level of risdiplam in blood of the neonatal subject is less than about 100 ng/mL. In embodiments, the second amount of risdiplam is administered to the neonatal subject within a time period of delivery of the neonatal subject (e.g., within 30 days after birth) if the level of risdiplam in cord blood of the neonatal subject at about the time of delivery corresponds to a defined mean safe exposure in humans, e.g., an area under the curve [AUCₛₛ] of 2000 ng/h/mL as described in the Prescribing Information for Evrysdi®.

In embodiments, the second amount of risdiplam is a therapeutically effective amount of risdiplam. In embodiments, the therapeutically effective amount of risdiplam is equal to or less than 0.15 mg/kg of the neonatal subject. In embodiments, the therapeutically effective amount of risdiplam is 0.15 mg/kg of the neonatal subject. In embodiments, administering the second amount of risdiplam to the neonatal subject occurs daily. In embodiments, administering the second amount of risdiplam to the neonatal subject occurs orally. In embodiments, the second amount of risdiplam is administered to the neonatal subject as an oral dose of up to 0.15 mg/kg/day. In embodiments, the administration of risdiplam to the neonatal subject at a daily oral dose of up to 0.15 mg/kg occurs during a time period of from delivery of the neonatal subject to about 2 months after delivery of the neonatal subject.

In embodiments, the second amount of risdiplam is administered to the neonatal subject as a daily oral dose of 0.20 mg/kg during a time period of about 2 months to about 2 years after delivery of the neonatal subject.

In embodiments, the neonatal treatment cycle comprises a first administration of risdiplam to the neonatal subject at a daily oral dose of 0.15 mg/kg during a time period of from delivery of the neonatal subject to about 2 months after delivery of the neonatal subject followed by a second administration of risdiplam to the neonatal subject at a daily oral dose of 0.20 mg/kg during a time period of about 2 months to about 2 years after delivery of the neonatal subject. In embodiments, the first administration of risdiplam from the daily oral dose of 0.15 mg/kg to the second administration of risdiplam at a daily oral dose of 0.20 mg/kg is continuous. In embodiments, the first administration of risdiplam at a daily oral dose of 0.15 mg/kg and the second administration of risdiplam at a daily oral dose of 0.20 mg/kg is sequential.

In embodiments, the SMN2 splice modification therapy comprises administering to the neonatal subject an amount of nusinersen or SPINRAZA®. In embodiments, the amount of the nusinersen is about 12 mg/5 mL (2.4 mg/mL). In embodiments, the amount of the nusinersen is administered to the neonatal subject via a single dose by intrathecal administration. In embodiments, initiation of the administration to the neonatal subject of the SMN2 splice modification therapy (such as nusinersen) as the second SMA therapy occurs with at least one loading dose. In such embodiments, the initiation of such SMN2 splice modification therapy occurs with up to four loading doses, where a first, a second, and a third loading dose are administered to the neonatal subject regularly at a time interval. In embodiments, the time interval is a fourteen-day time interval. In embodiments, a fourth loading dose is administered at a time period after the first three loading doses. In embodiments, the time period is about 30 days after the third loading dose. In embodiments, administration of the SMN2 splice modification therapy comprises a maintenance dose administered at a time period subsequent to the loading doses. In embodiments, the time period is once every four months after the fourth loading dose.

In embodiments, the neonatal treatment cycle comprises administering the second SMA therapy to the neonatal subject, where the second SMA therapy is the SMN expressing gene therapy. In embodiments, the SMN expressing gene therapy is an adeno-associated viral vector- based gene therapy. In embodiments, the adeno-associated viral vector-based gene therapy is onasemnogene abeparvovec-xioi or onasemnogene abeparvovec or Zolgensma®. In embodiments, the SMN expressing gene therapy comprises administering to the neonatal subject an amount of onasemnogene abeparvovec. In embodiments, the amount of the onasemnogene abeparvovec is a therapeutically effective amount of 1.1×10¹⁴ vector genomes (vg) per kg of body weight. In embodiments, the amount of the onasemnogene abeparvovec is administered via a single intravenous infusion that occurs once every time period. In embodiments, the time period comprises about sixty minutes. In embodiments, at a time period prior to initiation of the administration of the amount of onasemnogene abeparvovec administration of an amount of systemic corticosteroids occurs. In embodiments, the time period is one day. In embodiments, the amount of systemic corticosteroids is equivalent to oral prednisolone at about 1 mg/kg of body weight. In embodiments, one day prior to initiation of the administration of the amount of onasemnogene abeparvovec the administration of the amount of systemic corticosteroids occurs once daily. In embodiments, the administration of the amount of systemic corticosteroids is terminated after a time period has elapsed. In embodiments, the time period is thirty days.

In embodiments, the second SMA therapy is combined with other therapies, such as a muscle enhancing therapy. As defined herein, a “muscle enhancing therapy” is any therapy that enables muscles (such as skeletal muscles) to increase in size (muscle growth) and/or in strength. In embodiments, the muscle enhancing therapy is a myostatin inhibitor. In embodiments, the myostatin inhibitor is an anti-myostatin antibody designed to target skeletal muscles and in particular, the myostatin pathway. Without being bound by any particular theory, it is believed that inhibiting myostatin may help muscles grow in size and strength and/or lead to an improvement in motor function. In embodiments, the myostatin inhibitor is apitegromab (SRK-015) an anti-promyostatin monoclonal antibody. In embodiments, the myostatin inhibitor is Taldefgrobep alfa (BHV2000) an anti-promyostatin monoclonal antibody. In embodiments, the myostatin inhibitor is GYM329 (Roche), a recycling and antigen-sweeping monoclonal anti-myostatin antibody. In embodiments, the myostatin inhibitor is BIIB110 (formerly ALG801) (Biogen), a recombinant protein acting as an ActRIIB (activin receptor type-2B) ligand trap. See WO2016098357, WO2017104783, WO2023057404, and WO2017049011, the entire contents of which are hereby incorporated by reference in their entirety. In embodiments, administration of the myostatin inhibitor to the neonatal subject is initiated at least six months from the date of delivery of the fetal subject as the neonatal subject. The myostatin inhibitor may be administered within at least the first day, the first week, or the first month of delivery of the fetal subject as the neonatal subject. In embodiments, the myostatin inhibitor is administered within about 3 to 6 months of delivery from the carrier. In embodiments, the myostatin inhibitor is administered after the initiation of administration of the second SMA therapy. In embodiments, the myostatin inhibitor is administered concurrently with the initiation of administration of the second SMA therapy.

In embodiments, the methods comprise determining when the neonatal subject who received the carrier treatment cycle as the fetal subject is in need of further treatment such as the neonatal treatment cycle and/or to evaluate the clinical response of the neonatal subject to the disclosed therapy. Disclosed testing and parameters measured to assess clinical response such as biomarker, electrophysiological and neurophysical parameters described herein can be measured in relation to the natural history of SMA or the natural history of SMA in the human subject. Accordingly, a complete or partial clinical response can be concluded based on a measured parameter that demonstrates an improvement from the natural disease course, maintenance of the condition or cessation of decline in the natural disease course, or non-inferior decline in the natural disease course. In embodiments, the natural history of SMA is in accordance with data from a reference study. In embodiments, the reference study is the NURTURE clinical trial (ClinicalTrials.gov ID NCT02386553) A Study of Multiple Doses of Nusinersen (ISIS 396443) Delivered to Infants With Genetically Diagnosed and Presymptomatic Spinal Muscular Atrophy. In embodiments, the reference study is the RAINBOWFISH clinical trial (ClinicalTrials.gov ID NCT03779334) A Study of Risdiplam in Infants With Genetically Diagnosed and Presymptomatic Spinal Muscular Atrophy.

Treatment eligibility and/or clinical response of the neonatal subject to the carrier treatment cycle may be evaluated upon delivery of the fetal subject and/or upon cessation of the carrier treatment cycle. Eligibility of the neonatal subject for the neonatal treatment cycle may be determined by a treating physician using a clinical response evaluation tool, a neurophysiology assessment, evaluation of biomarkers, histological clearance evaluation, and/or at a physician's discretion. The clinical response may include a complete response, a partial response, a stable disease, or a progressive disease. The clinical response may be evaluated based on numerous criteria, including an amount of risdiplam exposure to the fetal subject based on a sample of amniotic fluid or fetal cord blood, a complete blood count (CBC) screening, a comprehensive metabolic panel (CMP), a urine analysis (UA), and detected neurological issues or risdiplam-specific adverse events (e.g., based on a physical examination of the neonatal subject). A neonatal subject who does not achieve complete response after completion of the carrier treatment cycle but achieves partial response, or demonstrates stable or progressive disease, may, at the treating physician's discretion, continue to receive further treatment including the neonatal treatment cycle.

In some embodiments, SMA biomarkers, such as SMN protein level, CMAP amplitude and neurofilament levels in the neonatal subject, may be used to evaluate the efficacy of risdiplam in neonatal subjects treated in utero. CMAP is an objective measure of the electrophysiologic output from the total output of all motor units that supply a particular muscle following supramaximal stimulation of the innervating nerve. CMAP may be used to monitor disease progression in SMA, as it is considered as a surrogate of motor neuron loss and correlates with disease severity, functional status, SMN2 copy number, and age, with a significant decline shown in CMAP amplitude in neonatal subjects with Type 1 SMA over the first few months of life. See Kolb (2016) Ann Clin Transl Neurol 3(2):132-45; Finkel (2013) Neuromuscul Disord 23(2):112-5; Lewelt (2010) Muscle Nerve 42(5):703-8; and Swoboda (2005) Ann Neurol 57(5):704-12. Moreover, CMAP amplitude may be used to determine disease onset in pre-symptomatic neonatal subjects. See Lee (2022) Neurology 99(14):e1527-37; Weng (2021) Genet Med. 23(2):415-20; and Vill (2019) J Neuromuscul Dis 6(4):503-15. CMAP amplitude correlates with functional motor scores (see Kolb (2017) Ann Neurol 82(6):883-91) and is predictive of walking status at 2 years of age (see Kariyawasam (2023) Lancet Child Adolesc Health 7(3):159-70).

In some embodiments, when the fetal subject is delivered as the neonatal subject, the CMAP amplitude of the neonatal subject may be assessed at a time period from delivery from the carrier, such as one week from delivery from the carrier, two weeks from delivery from the carrier, one month from delivery from the carrier, two months from delivery from the carrier, six months from the delivery from the carrier, and/or twelve months from delivery form the carrier, etc. to predict the efficacy of in utero treatment with risdiplam.

In some embodiments, the fetal subject diagnosed by genetic testing as having SMA has elevated neurofilament levels in at least one body fluid, such as in plasma or cerebrospinal fluid (CSF). When the fetal subject is delivered as the neonatal subject, the neonatal subject may be determined to have an elevated neurofilament level in at least cord blood. Neurofilaments are neuron-specific and are composed of four subunits, including neurofilament light chain (NfL) and heavy chain (NfH). Following neuroaxonal damage as can occur in SMA, neurofilaments are released into the interstitial fluid, the CSF and peripheral blood. Elevated levels of NfL and pNfH related to motor neuron damage have been observed in subjects with SMA compared with healthy controls. See Paris (2023) CPT Pharmacometrics Syst Pharmacol 12(2):196-206; Alves (2021) Mol Ther Methods Clin Dev. 23:524-38; and Darras (2019) Ann Clin Transl Neurol 6(5):932-44. Neurofilaments serve as a potential blood biomarker for SMA and neurofilament light chain levels in serum (sNfL) in treatment-naïve SMA patients with 2 SMN2 copies are higher than in those with >2 SMN2 copies. See Nitz (2021) Ann. Clinc. Transl. Neurol. 8(10):2013-2024. Moreover, NfL and pNfH levels are elevated in neonatal subjects with SMA in the first months of life as compared to healthy controls. Those levels are inversely correlated with maximum ulnar CMAP negative peak amplitude values. See Alves (2021) Mol Ther Methods Clin Dev. 23:524-38. Further, plasma concentrations of pNfH in neonatal subjects with SMA younger than seven months of age treated with SMA disease-modifying treatment (DMT) decreased faster and to a greater magnitude compared to a sham control arm. See Darras (2019) Ann Clin Transl Neurol 6(5):932-44. In some embodiments, the pNfH levels of the neonatal subject may be assessed at a time period from delivery from the carrier, such as delivery from the carrier, at the first month from delivery from the carrier, and/or at the second month from delivery from the carrier, etc. to predict efficacy of in utero treatment with risdiplam.

As described herein, a complete response to various assessments may include, for example, achieving certain developmental milestones evaluated according to metrics described in Table 1.

TABLE 1

| Candidate | | |
|---|---|---|
| Outcomes | Type 1 | Type 2/3 |
| Time to event | Time to death or | Time to death or |
| | required ventilation | required ventilation |
| | for >16 hours/day | for >16 hours/day |
| Physical function | Children's Hospital | Gross Motor Function |
| | of Philadelphia Infant | Measure (GMFM) |
| | Test of | Hammersmith |
| | Neuromuscular | Functional Motor |
| | Disorders (CHOP | Scale - Expanded |
| | INTEND) | (HFMSE) |
| | Test of Infant Motor | Motor Function |
| | Performance (TIMP) | Measure-32 (MFM-32); |
| | Bayley Scales of | Motor Function |
| | Infant and Toddler | Measure-20 (MFM-20) |
| | Development, 3^{rd} | Upper Limb Module |
| | Edition (BSID-III) | (ULM); Revised |
| | Bayley Scales of | Upper Limb Module |
| | Infant and Toddler | (RULM) |
| | Development, 4^{th} | Revised Hammersmith |
| | Edition (BSID-IV) | Scale (RHS) |
| | Test of Infant Motor | Motor Function |
| | Performance | Measure-32 |
| | Screening Items | (MFM32)* |
| | (TIMPSI) | Six-minute walk test |
| | Alberta Infant Motor | (6MWT, ambulant |
| | Scale (AIMS) | patients only) |
| | Hammersmith Infant | |
| | Neurological | |
| | Examination (HINE); | |
| | Hammersmith Infant | |
| | Neurological | |
| | Examination-Part 2 | |
| | (HINE-2) | |
| | World Health | |
| | Organization (WHO) | |
| | motor milestones | |
| Patient- or | N/A - Not possible to | Pediatric Quality of |
| caregiver- | reliably assess | Life Inventory ™ |
| reported | | (PedsQL) |
| quality of life | | Pediatric Evaluation |
| | | of Disability |
| | | Inventory Computer |
| | | Adaptive Test |
| | | (PEDI-CAT) |
| | | Assessment of |
| | | Caregiver Experience |
| | | with Neuromuscular |
| | | Disease (ACEND) |
| Pulmonary | Frequency of | Spirometry, e.g., |
| | respiratory infections | forced vital capacity |
| | Pulse oximetry and | (FVC), forced |
| | end-tidal CO₂ | expiratory volume in |
| | Sleep evaluation and | 1 second (FEV1) |
| | nocturnal | Noninvasive: Peak |
| | hypoventilation | cough flow (PCF); |
| | Respiratory muscle | maximal |
| | fatigue | inspiratory/expiratory |
| | Respiratory | pressure (MIP/MEP); |
| | inductance | maximal |
| | plethysmography | inspiratory/expiratory |
| | (RIP) | pressure (PIₘₐₓ/Peₘₐₓ); |
| | Time to permanent | spontaneous nasal |
| | ventilation** | inspiratory pressure |
| | | (SNIP) |
| Electrophysiology | Compound muscle | Compound muscle |
| and myography | action potential | action potential |
| | (CMAP) amplitude | (CMAP) amplitude |
| | Motor unit number | Motor unit number |
| | estimation (MUNE) | estimation (MUNE) |

*MFM32 is a thirty-two-item clinician-reported outcome measure used to assess the functional abilities of subjects with neuromuscular diseases, including those with SMA.
**The time to permanent ventilation includes tracheostomy or ventilation [bilevel positive airway pressure] for ≥16 hours per day continuously for >3 weeks or continuous intubation for >3 weeks, in the absence of, or after the resolution of, an acute reversible event, as assessed with the use of the Kaplan-Meier method.

A complete response may include the neonatal subject being evaluated according to one or more of the above outcomes, and the resulting evaluation falling within what is expected or considered normal for a neonatal subject without SMA. For example, a neonatal subject is considered small for its gestational age if it is at or below the third percentile of normal range for weight-for-age, length/height-for-age, and weight-for-length/height of age/visit and head circumference-for-age of age/visit based on the World Health Organization (WHO) Child Growth Standards. Accordingly, a complete response may include evaluating the neonatal subject for one or more of weight-for-age, length/height-for-age, and weight-for-length/height of age/visit and head circumference-for-age of age/visit based on the World Health Organization (WHO) Child Growth Standards, and finding that the neonatal subject is above the third percentile or normal range. As another example, a neonatal subject is considered to have a cognitive deficit if it has a scaled score below 1.5 standard deviation of chronological reference standard as measured by the Bayley Scales of Infant and Toddler Development, Third Edition (BSID-III) Cognitive Scale. Accordingly, a complete response may include evaluating the neonatal subject for cognitive deficit as measured by the Bayley Scales of Infant and Toddler Development, Third Edition (BSID-III) Cognitive Scale, and finding that the scaled score is not below 1.5 standard deviation of chronological reference. A complete response may include demonstrating normal, age-appropriate tone, posture, strength, activity, reflexes, feeding and breathing patterns based on a neurological exam. A complete response may include a normal birth weight for their gestational age, based on birth weight charts. A complete response may include being above the third percentile of normal range for head circumference-for-age of age/visit based on the WHO Child Growth Standards (WHO 2019). In some embodiments, administration of risdiplam to the carrier during gestation may increase the chances of being evaluated as normal based on one or more of the criteria in Table 1, compared to if the carrier were not administered risdiplam during gestation. Such an increase in the chances of achieving said evaluation, up to and including a normal response, may be evaluated or demonstrated by comparing neonatal subjects with SMA who were administered risdiplam during gestation through their carrier, to neonatal subjects with SMA who were not administered risdiplam during gestation through their carrier.

The clinical response may be assessed at any suitable time or times during or after the disclosed treatment. In embodiments, the clinical response is determined after the completion of the carrier treatment cycle. In embodiments, the clinical response is determined after the completion of the neonatal treatment cycle. In embodiments, the clinical response is determined after the completion of the carrier and the neonatal treatment cycles. In embodiments, the clinical response is determined after the completion of the carrier treatment cycle, but before an initiation of the neonatal treatment cycle.

In certain embodiments, the neonatal treatment cycle is not required, and the treatment ends after the carrier treatment cycle. In some embodiments, the neonatal treatment cycle is required. For example, for the neonatal subjects who do not achieve complete response after the carrier treatment cycle as fetal subjects, or for the neonatal subjects who may benefit from more than one treatment cycle, the neonatal treatment cycle may provide effective treatment. In some embodiments, the neonatal subject who receives the neonatal treatment cycle achieves a complete response after the carrier treatment cycle as the fetal subject. In some embodiments, the neonatal subject who receives the neonatal treatment cycle did not achieve a complete response (e.g., only achieves a partial response or demonstrates stable or progressive disease) after the carrier treatment cycle as the fetal subject. In embodiments, an eligible neonatal subject for the additional treatment cycle shows the clinical response after the carrier treatment cycle, but before the neonatal treatment cycle. In embodiments, an eligible neonatal subject shows partial clinical response. In embodiments, an eligible neonatal subject shows stable disease. In embodiments, an eligible neonatal subject shows progressive disease. In embodiments, an eligible neonatal subject may be asymptomatic, but may still require the second SMA therapy due to the progressive nature of SMA (e.g., to prevent disease manifestation or to maintain the gains from fetal therapy).

In embodiments, the treatment achieves a beneficial clinical response (e.g., the partial response or the stable disease) for at least one of the symptoms after the carrier treatment cycle, but before the neonatal treatment cycle. In embodiments, the disclosed treatment does not achieve the complete response (e.g., achieves the partial response or the stable disease) in at least one of the symptoms after the carrier treatment cycle, but achieves the complete response in at least one of the symptoms after the neonatal treatment cycle. In embodiments, the disclosed treatment achieves a beneficial clinical response (e.g., the complete response, the partial response, or the stable disease) in at least one of the symptoms.

### Examples

The following Examples are set forth to enable this disclosure to be more fully understood. It should be understood that these examples are for illustrative purposes only and are not to be construed as limiting this disclosure in any manner.

### Example 1. A Single Patient Investigational Plan to Assess the Safety and Efficacy of Risdiplam in the Treatment of SMA in a Fetal Patient

The fetal subject was considered eligible for inclusion in this study since all of the following criteria applied: (1) the fetal subject was diagnosed as having SMA by genetic testing, (2) the fetal subject was within its third trimester of development, (3) the gestation of the fetal subject was in a range of about twenty-eight weeks to about thirty-eight weeks gestation, (4) the fetal subject had no detectable functional copies of the SMN1 gene, and (5) the fetal subject had at least one copy of the SMN2 gene. In particular, genetic testing of the fetal subject confirming no copies of the SMN1 gene and two copies of the SMN2 gene, together with a family history of SMA, was highly predictive of Type 1 SMA. See Jones (2020) J. Neuromuscul. Dis. 7:33-40; and Glascock (2018) J. Neuromuscul. Dis. 5(2):145-158. The fetal subject would have been excluded from this study if: (1) the fetal subject was within the first or second trimester of development, (2) the fetal subject had at least one copy of the SMN1 gene, or (3) the fetal subject had less than one copy of the SMN2 gene.

The fetal subject assigned to this treatment plan received risdiplam through the carrier after all basic organ systems of the fetal subject had substantially completed fundamental embryogenesis in its third trimester of development in a range of from about thirty-two and a half weeks to its delivery from the carrier at about thirty-eight and a half weeks gestation. During this period, risdiplam was administered to the fetal subject through a carrier treatment cycle in which a single daily dose of 5 mg of risdiplam was administered orally to the carrier of the fetal subject.

The carrier treatment cycle was terminated when the carrier of the fetal subject delivered the fetal subject as the neonatal subject. The carrier treatment cycle could have also been terminated if an adverse change in the biophysical profile of the fetal subject was detected. The carrier treatment cycle could have also been terminated if a decline in fetal growth occurred by more than 10% in an expected growth rate from a baseline. Ten percent is chosen to acknowledge the variability in fetal ultrasound predictions of fetal weight (using the Hadlock A formula, with a commonly accepted error rate of 5%). See Milner (2018) Ultrasound 26(1):32-41. The carrier treatment cycle could have also been terminated if a ventricular size, cardiac anatomy, renal or liver changes occurred to fetal organs. Further, the carrier treatment cycle could have also been terminated if non-manageable medical problems arose with respect to the carrier, including, for example, pregnancy-induced hypertension/pre-eclampsia. None of these events occurred.

Risdiplam trough drug levels were obtained from the carrier and the subject as the neonatal subject at time points represented in Table 2A and Table 2B from 32.5 weeks gestation prior to administration of risdiplam to the carrier through 43 days post-delivery. The risdiplam drug levels detected in plasma (ng/mL) of the carrier and the neonatal subject were determined by liquid chromatography-tandem mass spectroscopy (LC-MS/MS). Table 2B is a continuation of Table 2A.

TABLE 2A

| | Prenatal (weeks gestation, WG) | | | |
|---|---|---|---|---|
| | | 32.6 WG | | |
| | 32.5 WG | (23 hours | | |
| Timeline | (pre-risdiplam) | post-risdiplam) | 36 WG | 38 WG |
| Carrier | BLQ* | 5.27 ng/ml | 12 ng/ml | 14 ng/ml |

| | |
|---|---|
| Fetal subject | not measured |

*below limit of quantification

TABLE 2B

| | Birth | | Postnatal (days, d) | | | | |
|---|---|---|---|---|---|---|---|
| Timeline | 38.5 WG | | 8 d | 20 d | 29 d | 43 d | |
| Carrier | 15.6 | ng/ml V | not | 0.7 ng/ml | 0.6 ng/ml | 0.4 | ng/ml |
| | 5.09 | ng/ml AF | measured | | | | |

| | | | | | | |
|---|---|---|---|---|---|---|
| Neonatal subject | Cord | 0.6 ng/ml | not | not | 62 | ng/ml |

| | | | |
|---|---|---|---|
| 11.2 | ng/ml V | measured | measured |
| 10.4 | ng/ml A | | |

As set forth in Table 2A, the carrier steady state for the risdiplam level in plasma was about 14 ng/mL. Steady state plasma risdiplam levels in the carrier was reached by 3 weeks and remained generally stable, even as the amniotic fluid volume and volume of distribution changed. It remains possible that steady state was achieved sooner, as the actual drug level in the fetal subject cannot be ascertained but can be predicted using cord blood levels at the time of delivery as a proxy measure.

According to Table 2B, the amniotic fluid (AF) risdiplam level was about 33% of the carrier plasma venous (V) risdiplam level, and the cord venous (V) and arterial (A) plasma risdiplam levels in the fetal subject was about 69% of the carrier plasma risdiplam drug level.

The neonatal subject drug elimination half-life from cord blood and day 8 post-delivery (d8) samples was 46 hours.

Safety considerations need to be a priority to avoid overly exceeding the risdiplam exposure of a mean ssAUC₀₋₂₄ of 2000 ng-h/mL. Consideration was given to the potential that the neonatal subject liver might be relatively immature, and therefore decreased metabolism of the hepatically metabolized risdiplam compared to older subjects, as well as any transient exposure of the nursing neonatal subject to additional drug via breast milk.

The elimination half-life calculated for the neonatal subject suggested that initiation of the risdiplam in the neonatal subject need not be delayed, and the neonatal subject was fed formula, thus precluding additional drug administration via breast milk. Since it was determined that a level of the risdiplam in the cord blood of the neonatal subject was less than about 100 ng/mL or about 0.1 mg/L when the neonatal subject was bom, the neonatal treatment cycle was initiated. The neonatal treatment cycle began 8 days after birth (d8) and included administering risdiplam to the neonatal subject at a single daily dose of 0.15 mg/kg.

The trough drug level in the neonatal subject on d43 (62 ng/ml) after 35 days on oral treatment at 0.15 mg/kg/day, was similar to reference data. See Baranello (2021) N. Engl. J. Med. 384:915-923. In the reference study, a first group of four infants were each treated with a final dose of risdiplam at 12 months of treatment of 0.08 mg per kilogram of body weight per day and a second group of seventeen infants were each treated with a final dose of risdiplam at 12 months of treatment of 0.2 mg per kilogram per day. See Baranello (2021) N. Engl. J. Med. 384(10):915-923. The risdiplam drug level in plasma of the neonatal subject was estimated to be similar to the exposure observed in the reference study.

A prenatal study schedule and administration schedule of risdiplam is summarized in Table 3.

TABLE 3

| | | | | | Day of |
|---|---|---|---|---|---|
| | | | Weekly | Biweekly | delivery of |
| | | | during | during | the fetal |
| | | | pregnancy | pregnancy | subject as |
| | | | of the | of the | the neonatal |
| | Day 1 | Day 2 | carrier | carrier | subject |
| Pre-dose blood collection | X | | | | |
| Risdiplam dosing* | X | | | | |
| Post-dose blood collection for | | X | | | |
| risdiplam pharmacokinetics | | | | | |
| (PK) and pharmacodynamics (PD) | | | | | |
| OBGYN monitoring (SOC) | | | X1 | | |
| Interim PK/PD collection and | | | | X2 | |
| safety labs for the carrier | | | | | |
| Cord blood and amniotic | | | | | X |
| fluid collection | | | | | |
| Interim adverse events (AE) | | | X | | |
| monitoring | | | | | |

*The risdiplam dosing occurred once daily throughout the study.

As shown in Table 3, “X1” occurred weekly or could have occurred more frequently as determined by a physician. X1 included routine monitoring of the carrier for the third trimester, specifically with regards to development of hypertension and features of pre-eclampsia or HELLP syndrome, with related blood and urine testing. X1 also included monitoring nausea, vomiting, or dehydration in the carrier and placental integrity and amniotic fluid volume. X1 further included fetal monitoring for fetal growth, organ system development, and fetal well- being and any signs of organ maldevelopment, especially in the brain, heart, kidney, and liver. A baseline pre-treatment full anatomical ultrasound study and growth scan were performed and subsequent ultrasonography occurred at three and six weeks before delivery of the fetal subject as the neonatal subject.

Also in Table 3, “X2” included safety labs for the carrier, such as the CBC screening, the CMP screening, and the UA, to assess potential toxicity from risdiplam, which were collected before dosing and then prior to delivery. The safety labs were collected at the baseline before dosing, then every 3-4 weeks up to delivery, including pre-operatively prior to the scheduled cesarean section. More frequent testing may occur if clinically indicated.

In addition, in Table 3, blood samples for risdiplam PK and PD were collected from the carrier prior to the first dose of the risdiplam and 24-hours post- dose of the risdiplam. PK/PD samples were collected from the carrier every two weeks for up to six weeks after delivery to assess risdiplam clearance. At the time of delivery, samples of umbilical cord blood and amniotic fluid were collected to assess risdiplam exposure to the fetal subject in utero. Collection of the cord blood and the amniotic fluid could have also included collection of the cord blood and the amniotic fluid during pregnancy of the carrier if an amniocentesis was deemed to be needed.

A neonatal study schedule and administration schedule of risdiplam is summarized in Table 4.

TABLE 4

| | Approximately | Weekly after | | | |
|---|---|---|---|---|---|
| | one week after | delivery of | | | Six |
| | delivery of the | the fetal | Two | Four | weeks |
| | fetal subject as | subject as the | weeks | weeks | after |
| | the neonatal | neonatal | after | after | delivery |
| | subject | subject | delivery | delivery | (EOS) |
| Physical/neurological | X | | | | X |
| examination of the | | | | | |
| neonatal subject | | | | | |
| Post-delivery PK/PD | | | X | X | X |
| collection of the | | | | | |
| carrier | | | | | |
| Post-delivery PK/PD | X5 | | | | X |
| collection and safety | | | | | |
| labs of the | | | | | |
| neonatal subject | | | | | |
| Interim AE | | X | | | |
| monitoring | | | | | |

In Table 4, the “X5” post-delivery PK/PD collection and safety labs of the neonatal subject included CBC screening, CMP screening, and UA, and such safety labs could be repeated if the results are deemed abnormal as compared to a healthy individual.

Table 5 summarizes results of the carrier and the fetal subject clinical assessments during the pregnancy of the carrier when exposed to risdiplam and in the neonatal subject for 6 weeks following delivery, for a minimum of 30 days following the last dose of risdiplam.

Specifically, Table 5 depicts the treatment emergent adverse events, seriousness of the event, intensity of the adverse event, relatedness of the adverse event to administration of the risdiplam, study term, and start and end dates for the adverse event the detected from this Example.

Each adverse event term is associated with a Medical Dictionary for Regulatory Activity (or MedDRA) term and the intensity is categorized based on the CTCAE grading. In Table 5, “Y” refers to yes and “N” refers to no and the intensity is measured from a lowest level of intensity or “I” to a highest level of intensity or “V.” “VSD” is a ventricular septal defect, “GERD” is gastroesophageal reflux disease, and “NICU” refers to neonatal intensive care unit.

TABLE 5

| | | | | | | | Days post- |
|---|---|---|---|---|---|---|---|
| | | | | | | | initiation of |
| | | | | Intensity | | Study | risdiplam |
| | Adverse | Adverse Event or | Serious | (CTCAE | | Term | administration |
| | Event # | MedDRA Term | (Y/N) | grading) | Relatedness | (Y/N) | to the carrier* |
| Carrier | 1 | Headache | N | I | Unlikely | N | 1 |
| | 2 | Nausea | N | I | Unlikely | N | 1 |
| | 3 | GERD | N | I | Unlikely | N | 2 |
| | 4 | Back pain | N | I | Unrelated | N | 44 |
| | 5 | Flushing | N | I | Unlikely | N | 6 |
| | 6 | Dysgeusia | N | I | Possibly | N | 11-25 |
| | 7 | Flushing | N | I | Unlikely | N | 15 |
| | 8 | Proteinuria | N | II | Unlikely | N | 24-43 |
| | | | | (bedrest) | | | |
| | 9 | Increased amniotic | N | II | Unlikely | N | 24-43 |
| | | fluid volume | | (bedrest) | | | |
| | 10 | Follicultis | N | I | Unlikely | N | 34-ongoing |
| | 11 | GERD | N | I | Unlikely | N | 35 |
| | 12 | Hypertension | N | II (Rx) | Unlikely | N | 44-55 |
| | 13 | Headache | N | I | Unlikely | N | 46-79 |
| | 14 | Abdominal | N | II (Rx) | Unrelated | N | 57-64 |
| | | pain | | | | | |
| Neonatal | 1 | Respiratory | Y | III | Unrelated | N | 43-45 |
| subject | | disorder | | (NICU) | | | |
| | | (hypoventilation) | | | | | |
| | 2 | Congenital disorder | Y | I | Unlikely | N | 46-ongoing |
| | | (VSD) | | | | | |
| | 3 | Weight gain | N | I | Possibly | N | 50-62 |
| | | poor | | | | | |

*The neonatal subject was born 43 days after the post-initiation of risdiplam administration to the carrier and risdiplam administration to the neonatal subject began 8 days post-birth.

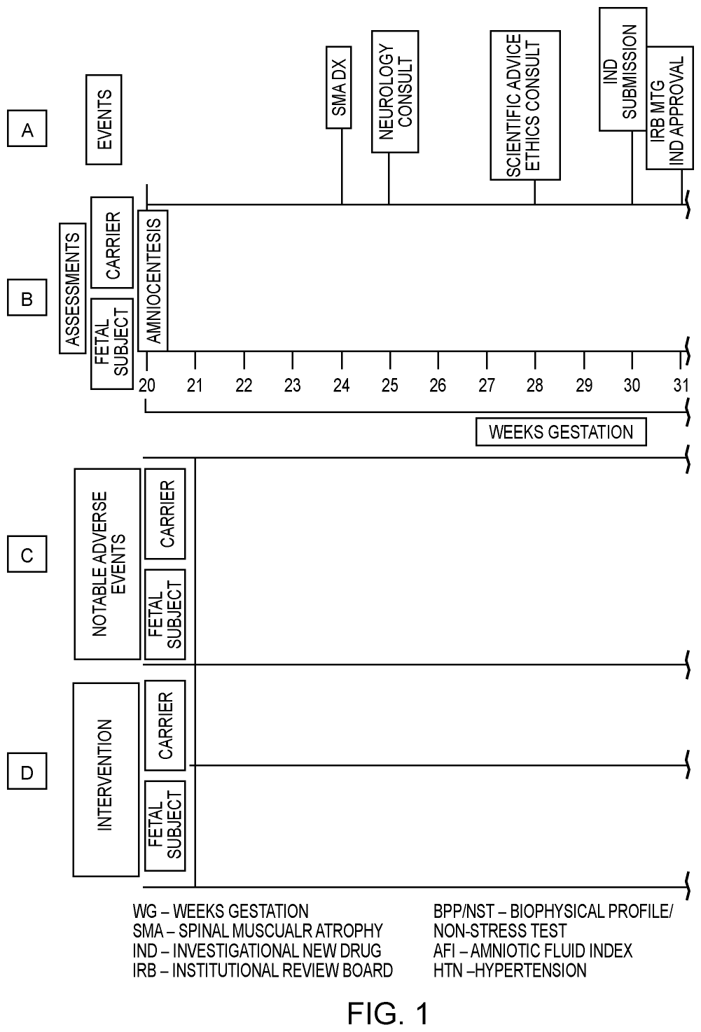

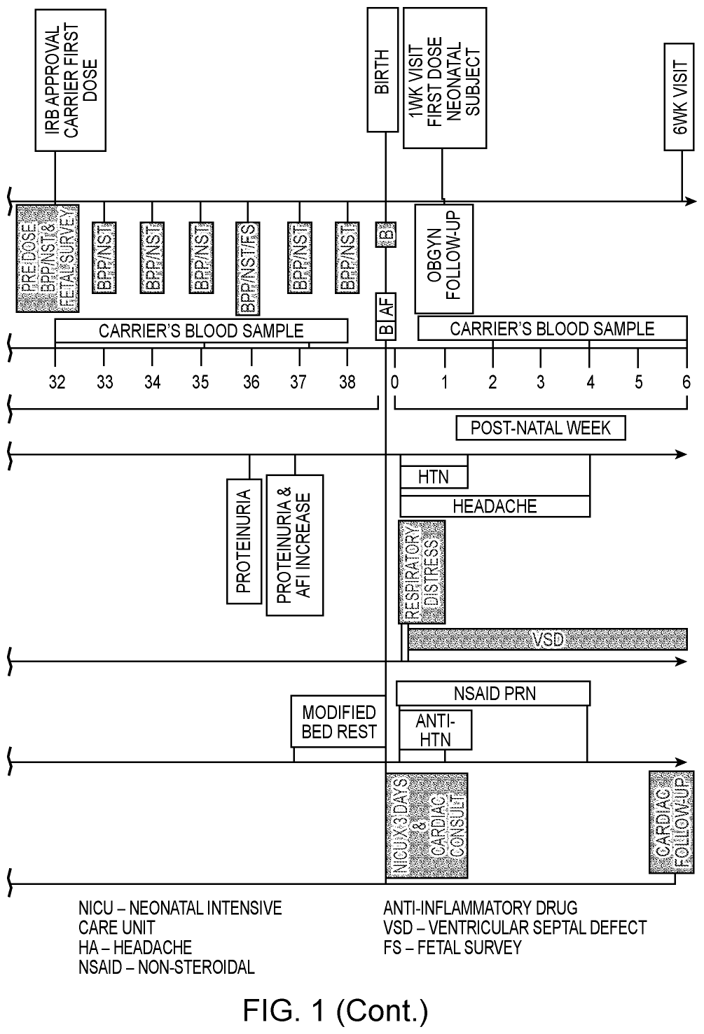

FIG. 1 includes charts associated with administering an amount of risdiplam to the carrier of the fetal subject and monitoring of the carrier and the fetal subject. Specifically, FIG. TA depicts events associated with the carrier and the fetal subject, FIG. 1B depicts assessments associated with the carrier and the fetal subject, FIG. 1C depicts notable adverse events associated with the carrier and the fetal subject, and FIG. 1D depicts intervention associated with the carrier and the fetal subject during the administration of the amount of risdiplam.

The postnatal course for the neonatal subject was complicated by retained fetal fluid in the lungs, presenting in the delivery room as mild tachypnea and hypoxemia. No hypoglycemia was identified. The neonatal subject received care in the neonatal intensive care unit (NICU) for 2 days, to include intubation and mechanical ventilation for 3 hours, quickly weaned to room air and needing no support by the third day post-delivery. As shown in Table 5, the neonatal subject's initial transition difficulty, attributed to retained fetal fluid in the lungs of the neonatal subject, and the VSD were unlikely related to risdiplam. Minor congenital cardiac defects were reported in 11% of 56 untreated individuals with SMA-1 and SMN2X2. See Rudnik-Schoneborn (2008) J. Med. Genet. 45:635-638. Intermittent sleepiness and slow weight gain the first 3 weeks were not felt to be excessive and also not likely to be drug-related. Thus, the carrier and the subject, as a fetal and neonatal subject, tolerated each procedure and treatment cycle with no drug-related adverse events.

Table 6 depicts further clinical assessments associated with this Example.

TABLE 6

| | Birth to 1 week | 1 week | 6 weeks | 18 weeks |
|---|---|---|---|---|
| General Physical | APGAR scores: 7 (1 | Heart murmur (VSD) | | |
| Exam | minute), 8 (5 minutes) | | | |
| | Respiratory distress | | | |
| Neurologic Exam | Normal, age- appropriate | Normal, age-appropriate tone, posture, | | |
| | tone, posture, strength, | strength, activity, reflexes, feeding and | | |
| | activity, reflexes, | breathing patterns | | |
| | feeding and breathing | | | |
| | patterns | | | |
| Mental status | Normal | Somnolent and fussy | | Normal after |
| | | 2 weeks to 2 months | | 2 months |
| Growth (weight | Birth weight 4.26 kg | Plateau in weight for | | Normal after |
| gain) | (99% for 39 WG) | 2 months | | 2 months |
| Ultrasound studies | normal DOL2 | normal | normal | normal |
| Neurosonogram | | | | |
| Muscle | | | | |
| CHOP INTEND | | 46 | 50 | 55^{#} |
| HINE-2 | | | | 8 |
| Bayley ™-4 | | | | Motor: 101 |
| | | | | Communication: 103 |
| | | | | Cognitive: 95 |
| | | | | Social: 95 |
| | | | | Adaptive |
| | | | | Behavior: 89 |
| Oral-motor | | 7 | 7 | 7 |
| assessment (OrSAT) | | | | |
| Electrophysiological | | | | |
| testing | | | | |
| Ulnar N CMAP | | 4.6 | 5.8 | 7.1 |
| (mV)* | | | | |
| Peroneal N CMAP | | 2.7 | 3.5 | |
| (mV) | | | | |
| MUNE (Ulnar N) | | 167 | 313 | 296 |
| Motor milestones | | | | Rolled prone to |
| | | | | supine at 12W2D |

*mean 5.5 mV for typically developing infants at 3 months) and ^{#}mean score in typically developing infants at 3.3 months = 50 (SD 10, range 32-62).

Surveillance labs during the carrier's pregnancy while on risdiplam were normal until proteinuria was detected at 36WG, prompting the recommendation for modified bed rest until the scheduled cesarean section at 39 6/7 WG. There were no signs of infection or gestational diabetes. Ultrasound assessments identified increased amniotic fluid volume at 37 WG.

The fetal ultrasound assessments were unremarkable and growth parameters followed normal trajectories. Biophysical profiles were normal at each time point. The laboratories of the neonatal subject performed while in the NICU were notable for transient hypercarbia without acidosis or hypoxemia. CBC, CMP, and U/A were normal, as shown in Table 6.

The post-partum course was complicated by transient hypertension, for which the carrier took an anti-hypertensive medication for 11 days. Delivery by scheduled repeat cesarean section at 38W6D WG was uncomplicated.

Following birth, the neonatal subject, approximately one week and again at approximately six weeks of age, was assessed using the Hammersmith Infant Neurological Exam (HINE), Bayley Scales of Infant and Toddler Development, 4^{th} edition (“Bayley™-4), the Children's Hospital of” Philadelphia Infant Test of Neuromuscular Disorders (CHOP INTEND), the Oral and Swallowing Abilities Tool (OrSAT), respiratory inductance plethysmography (RIP), a complete physical examination, anthropometric measurements (including vital signs, length, weight, head circumference and chest circumference), nerve conduction testing (specifically compound muscle action potential (CMAP) and motor unit number estimation (MUNE)), an ultrasound of the brain and muscles, blood and urine collection for CBC, CMP, U/A, and risdiplam PK/PD. The CBC, CMP, and U/A could be repeated at six weeks if the neonatal subject had not initiated any SMN-directed therapies, if there was a clinical indication upon physical examination, or if the one-week labs had clinically significant abnormal results.

The maximum score for the HINE-2 is 26 points, with a higher score reflecting a higher function, respectively. The neonatal subject had a score of 8 which may indicate the motor abilities of the neonatal subject.

Bayley™-4 is a comprehensive assessment tool for determining developmental delays in high-risk neonatal subjects. See Bayley & Aylward (2019) Bayley Scales of Infant and Toddler Development (4th ed.) technical manual. Bloomington, MN: NCS Pearson. Bayley™-4 scores from an assessment of early childhood development. Specifically, Bayley™-4 tests developmental skills such as cognitive, language, and motor skills of the neonatal subject. The scores represent how well the neonatal subject performed in these areas as compared to a group of neonatal subjects in the same age range. The Bayley™-4 mean standard score is 100 and the standard deviation is 15. As shown in Table 6, the Bayley™-4 was administered at 18 weeks of age, with the standard scores of the neonatal subject falling within the normal range for motor composite, overall communication, cognitive, social and adaptive behavior.

In Table 6, “CHOP INTEND” refers to a test that provides information about how strong the neonatal subject's muscles are and how well the neonatal subject can control his/her muscles. During the CHOP INTEND examination, a health care provider measures 16 types of muscle movements, which include head control (keeping the head upright), elbow flexion and knee extension (bending the joints), arm and leg mobility, and handgrip. Each of the 16 motor skills is given a score from 0 to 4, where a score of 0 indicates that the subject cannot complete the movement, a midrange score of 1, 2, or 3 means the subject can partially perform the motor skill, and a score of 4 means that the subject can fully complete the movement on their own, without assistance. All of the CHOP INTEND scores add up to one total score, with the highest possible score for the CHOP INTEND test being 64. With a score of 50 at 6 weeks and a score of 55 at 18 weeks, the neonatal subject was considered to be normal.

“OrSAT” or “Oral and Swallowing Abilities Tool” is a tool specifically designed to record structured information on different aspects related to oral, swallowing and feeding abilities in patients with type 1 SMA. See Berti (2022) Arch. Dis. Child. archdischild-2022-323899. Specifically, OrSAT includes several questions, where the responses are graded using a scoring system: each item is scored as 0 or 1 depending if the patient is able or unable to perform a given activity. Since some items are age-dependent, the number of items and the maximal score increases with an increasing age. If the neonatal subject younger than 6 months of age who cannot be assessed on swallowing semisolids or solids, the maximum score is 7, whereas if the neonatal subject is between 6 and 9 months of age, who can be assessed on this criteria, the maximum score is 10. If the neonatal subject is 10 months of age or older and can be assessed for all food consistencies, including solids, the maximum total score is 12. See Berti (2021) J. Neuromuscul. Dis. 8(4):589-601. As shown in Table 6, the OrSAT score of 7/7 is considered normal.

The electrophysiological testing described in this Example can serve as a prognostic biomarker. See Pino (2021) Biomark. Insights. 16:11772719211035643. Electrophysiological studies were performed using standard technique on a Natus UltraPro S100 instrument, with Natus pre-gelled disposable surface electrodes (#901350242) and standard settings for motor nerve conduction studies (LFF 10 Hz and HFF 10 MHz). The right ulnar nerve was stimulated at the elbow and recorded from the abductor digiti minimi (ADM) and the peroneal (fibular) nerve was stimulated at the knee and recorded from the tibialis anterior (TA) muscle.

Electrophysiological measurements, such as the compound muscle action potential (CMAP) in millivolts (mV) and motor unit number estimation (MUNE), monitor the functional status of the motor unit pool and are particularly pertinent to motor neuron disorders. See Arnold (2014) Ann. Clin. Transl. Neurol. 1(1):34:44. Cross-sectional studies have shown that CMAP and MUNE correlate with other measures of motor function, clinical severity, and overall function. See Arnold (2014) Ann. Clin. Transl. Neurol. 1(1):34:44.

The CMAP response measures the output of the motor units supplying a particular muscle or group of muscles. The CMAP size is determined by the size and number of depolarized muscle fibers following supramaximal nerve stimulation. Three active electrode placements were used for each nerve tested and the maximal amplitude of the negative peak of the CMAP was used for analysis. As a reference study for the CMAP data provided in Table 6, an ongoing Phase 2, open-label study (NURTURE) aims to evaluate the safety and efficacy of nusinersen in preventing or profoundly attenuating the severity of SMA when initiated prior to the onset of symptoms. See De Vivo (2019) Neuromuscul. Disord. 29(11):842-856. In accordance with this reference study, CMAP amplitudes were mean (SD) 2.7 (1.5) and median (range) 2.3 (1.0-6.7) and NeuroNEXT (typically developing infants) was mean (SD) 5.5±2.0. See Kolb (2016) Ann. Clin. Transl. Neurol. 3(2):132-45.

Traditional ulnar CMAP scores are 5.5mv (+/−2) as reported in the NeuroNEXT study comparing SMA infants to typically developing infants. See Kolb (2016) Ann. Clin. Transl.

Neurol. 3(2):132-45. According to this reference study for SMA involving neonatal subjects with 2 copies of SMN2 (SMN2X2), the median peroneal CMAP is 3.20 mV, with the range being 1.1-9.7 mV, the mean being 2.69 mV, and the standard deviation being 1.516 mV. See De Vivo (2019) Neuromuscul. Disord. 29(11):842-856. When compared to the ulnar nerve CMAP for participants in the NURTURE study with SMN2XS (median 2.3 mV), the neonatal subject data of 4.6 mV at 1 week (2-fold higher) and 5.8 mV at 6 weeks (2.5-fold higher) suggest relative preservation of motor neurons in the neonatal subject. See De Vivo (2019) Neuromuscl. Discord. 29:842-856. These values are similar to the mean value of 5.5 mV reported in typically developing neonatal subjects with mean age 3.3 months. See Kolb (2016) Ann. Clin. Transl. Neurol. 3:132-145.

MUNE is an electrophysiological method that can measure the number of motor units supplying a particular muscle. It should be appreciated that the “N” of MUNE (ulnar N) in Table 6 refers to “nerve.” For pre-symptomatic SMA neonates, MUNE (ulnar N) ranges from 70-270. See Bromberg (2002) Muscle Nerve. 25(3):445-7; and Swoboda (2005) Ann Neurol. 57(5):704-12. In this Example, the MUNE was performed on the right ulnar nerve using the multipoint technique. A minimum of ten unique single motor unit potentials were captured with the mean value used for the calculation of the MUNE. Published data on MUNE in five pre-symptomatic neonates with SMA ranged from 70 to 270 suggests that the neonatal subject maintained a substantial pool of motor neurons at least to 6-weeks of age. See Swoboda, et al. (2005) Ann. Neurol. 57:704-712; and Bromberg (2002) Muscle Nerve 25:445-447.

Muscle ultrasound was performed using a 11-mHz-linear array transducer (60 mm width) in B-mode (GE Vivid E95; GE, Chicago, IL, USA) to evaluate for muscle fasciculation in the right and left biceps, the abductor digiti minimi muscle, the quadriceps, the transverse abdominal muscle, the gastrocnemius-soleus, the lumbar paraspinals and the tongue. Thickness of the quadriceps muscle was measured. These examinations were examined jointly by two experienced evaluators.

Table 7A and Table 7B presents SMN protein levels in whole blood (measured in ng/mL) in the carrier and the subject as the fetal subject and the neonatal subject of this Example, and reference values are shown in Table 7C below. Table 7B is a continuation of Table 7A from 32.5 weeks gestation prior to administration of risdiplam to the carrier through 43 days post-delivery. SMN protein levels in the whole blood were performed using an immunoassay developed by Roche Diagnostics on the Elecsys® platform.

TABLE 7A

| Timeline | Prenatal (weeks gestation, WG) | | | |
|---|---|---|---|---|
| (weeks | | 32.6 WG | | |
| gestation | 32.5 WG | (23 hours | | |
| or “WG”) | (pre-risdiplam) | post-risdiplam) | 36 WG | 38 WG |
| Carrier | 7.7 ng/mL | — | 6.3 ng/mL | 6.0 ng/mL |

| | |
|---|---|
| Fetal Subject | Not measured |

TABLE 7B
Timeline
(days

| post-birth | Birth | Postnatal (day post birth, d) | | | |
|---|---|---|---|---|---|
| or “d”) | 38.5 WG | d 8 | d 20 | d 29 | d 43 |
| Carrier | 4.0 ng/mL | — | 5.0 ng/mL | 5.1 ng/mL | 4.6 ng/mL |
| Neonatal | 9.4 ng/mL | 4.5 ng/mL | — | — | 6.1 ng/mL |
| Subject | (cord) | | | | |

As shown in Table 7A and Table 7B, the SMN protein level of the carrier declined 48% from prenatal treatment until delivery and then increased 15-28% in the postpartum period. The SMN protein level of the neonatal subject declined 52% from the cord blood level to d8, during which time no drug was administered, then increased 36% at d43 following drug initiation on d8.

The SMN protein level declines with age and it is theorized that the declining SMN protein levels in the carrier may reflect something similar during pregnancy. The SMN protein level in the neonatal subject at birth suggests a pharmacodynamic effect of prenatal treatment. The target blood level of SMN in the fetal subject required for optimal motor neuron survival and function during fetal development is unknown. Unlike the theoretical risk of SMN overexpression from gene therapy (e.g., onasemnogene abeparvove therapy) (see Van Alstyne (2021) Nat. Neurosci. 24(7):930-940), there is no such concern with risdiplam, as its effects are limited by the maximum possible shift in SMN2 pre-mRNA splicing to full inclusion of the Exon 7 in the transcript.

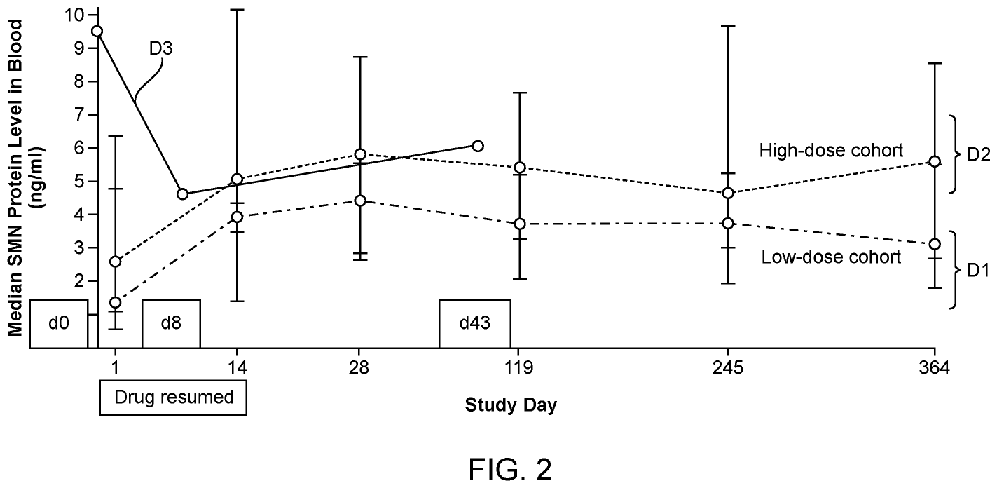

FIG. 2 depicts a graph comparing the SMN protein level in whole blood in Example 1 to a reference study with reference SMN protein levels provided in Table 7C.

TABLE 7C

| | Low dose | High dose |
|---|---|---|
| | (0.08 mg/kg/day) | (0.2 mg/kg/day) |
| Age at start | 6.9 (6.7-6.9) months | 6.3 (3.3-6.9) months |
| of treatment | | |
| Carrier | — | — |
| Reference | T0: 1.3 (0.6-4.8) | T0: 2.5 (1.1-6.4) |
| values* | 4 W risdiplam: | 4 W risdiplam: |
| (median, range) | 4.5 (2.6-5.6) | 5.9 (2.8-8.8) |
| | 12 M risdiplam: | 12 M risdiplam: |
| | 3.0 (1.8-5.5) | 5.7 (2.7-8.6) |

*See Baranello (2021) N. Engl. J. Med. 384 (10): 915-923.

In FIG. 2, the x- axis associated with the study day and the y-axis is associated with the median SMN protein level in whole blood (measured in ng/mL). Lines D1, D2, and D3 are depicted. Line D1 refers to the low dose amount of 0.08 mg/kg/day and Line D2 refers to the high dose of 0.2 mg/kg/day in Table 7C associated with the reference study. Line D3 depicts data points associated with this Example. As can be seen in FIG. 2, at d0, the prenatal SMN protein level in whole blood was about two times the reference amounts. At d8, the prenatal SMN protein level in whole blood declined about 52% when off risdiplam and at d43, the prenatal SMN protein level in whole blood was the median of the reference amounts.

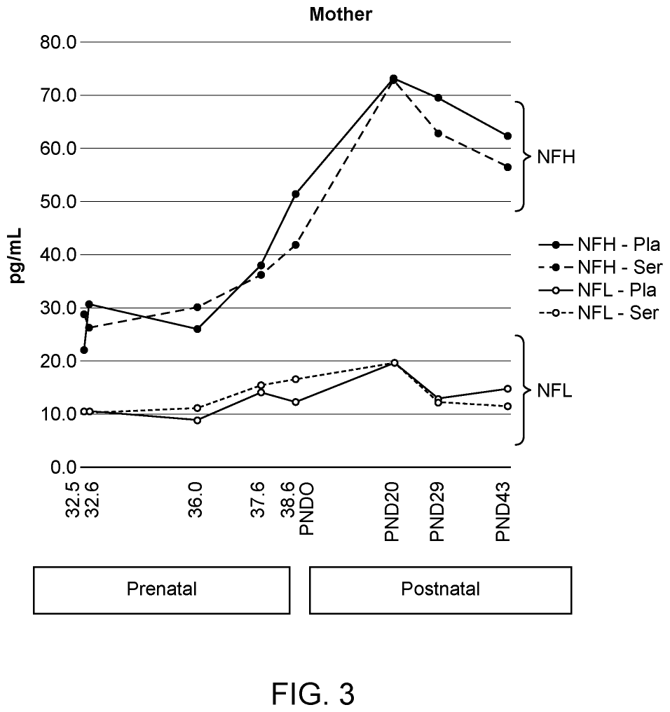

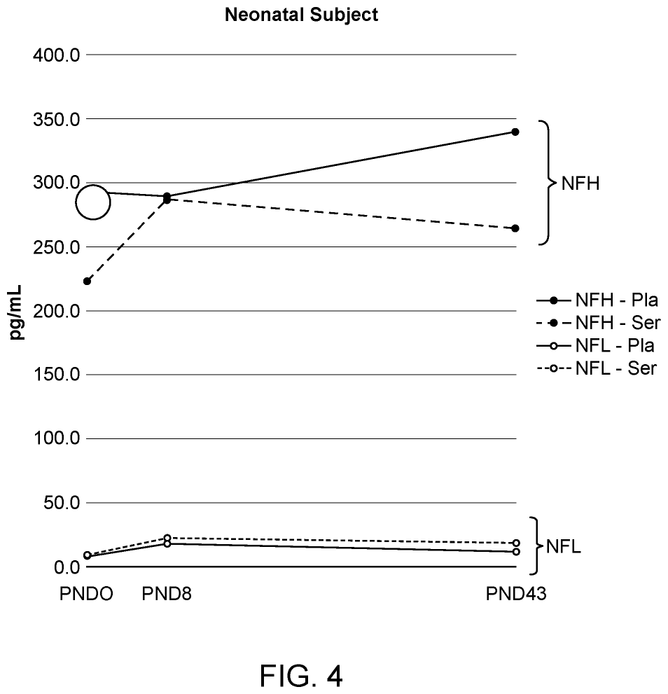

Neurofilament light and phosphorylated heavy chain assays were performed on plasma and serum samples using an ELISA assay, following the manufacturer's instructions (ProteinSimple, San Jose, CA, USA). Two runs were performed, each run having 3 aliquots run on the same plate and the mean value utilized. The average of the two runs was used for interpretation and in constructing FIG. 3 and FIG. 4.

FIG. 3 provides a graph depicting a mean neurofilament level in blood for the carrier on various dates, in accordance with at least some embodiments disclosed herein and FIG. 4 provides a graph depicting a mean neurofilament level in blood for the neonatal subject on various dates, in accordance with at least some embodiments disclosed herein.

The x-axis in FIG. 3 and FIG. 4 is associated with the date. The x-axis in FIG. 3 includes both prenatal dates (e.g., 32.5 WG or weeks gestation-37.6 WG) and postnatal dates or “PND” (e.g., d0-d43, referring to days post-birth of the fetal subject as the neonatal subject). The y- axis in FIG. 3 and FIG. 4 is associated with the mean neurofilament levels in blood (measured in pg/mL).

Further, in FIG. 3 and FIG. 4, “NF-L” refers to “neurofilament light chain,” which is a neuronal protein highly expressed in large caliber myelinated axons. NF-L levels increase in cerebrospinal fluid and blood proportionally to the degree of axonal damage in a variety of neurological disorders, including inflammatory, neurodegenerative, traumatic and cerebrovascular diseases. See Gaetani (2019) J. Neurol. Neurosurg. Psychiatry. 90(8):870-881. Additionally, “pNF-H” refers to the phosphorylated neurofilament heavy subunit (or phosphorylated NF-H). Further, as shown in FIG. 3 and FIG. 4, “Ser” refers to blood serum and “Pla” refers to blood plasma.

As seen in FIG. 3 and FIG. 4, the NF-L levels are low, with little change and are similar between the carrier and the neonatal subject. The pNF-H levels in the neonatal subject at delivery were about 10-fold higher in the neonatal subject as compared to the carrier. The post- partum pNF-H levels of the carrier increased approximately 2-fold during the 6-week observation period, with less relative change in NF-L levels. The neonatal subjects NF-L and serum pNF-H levels increased slightly from the cord blood sample to d8 then stabilized and the plasma pNF-H showed a transient decrease from birth to d8 then an increase to d43.

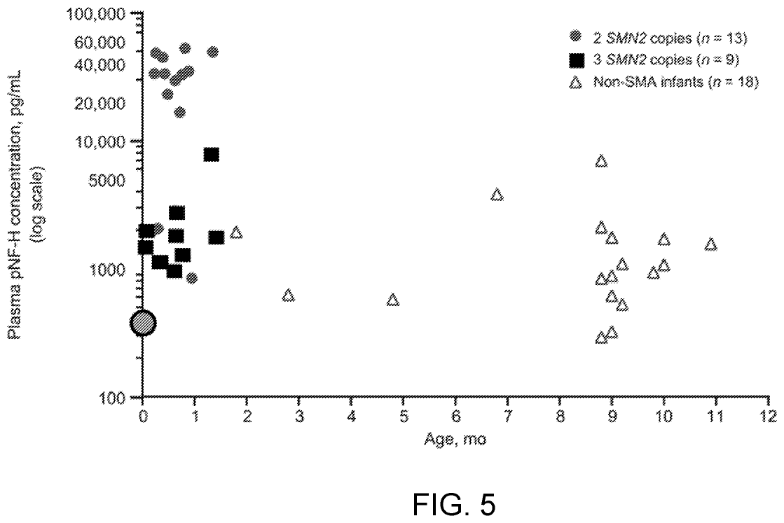

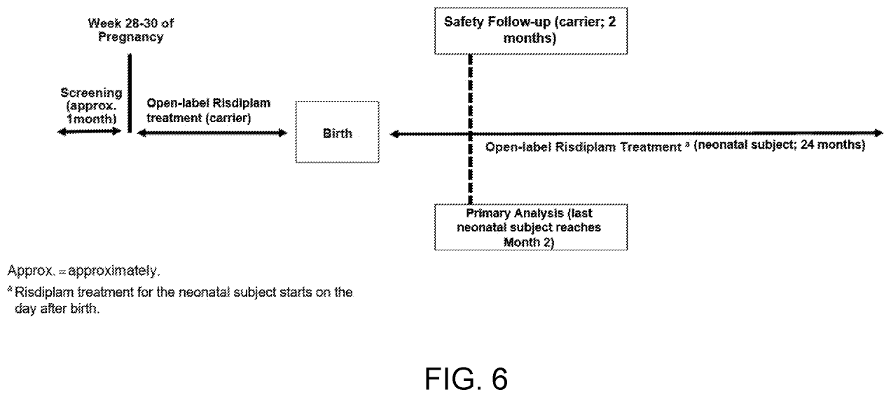

FIG. 5 depicts a graph of a reference study measuring plasma pNF-H concentration (pg/mL) levels at a baseline in NURTURE infants and infants<1 year of age without SMA, in accordance with at least some embodiments disclosed herein. See FIG. 6A of De Vivo (2019) Neuromuscul. Disord. 29(11):842-856. Specifically, pNF-H levels in FIG. 5 were evaluated using a pNF-H ELLA from ProteinSimple and 7.46 pg/mL was used as the imputed value if the pNF-H concentration was below the limit of quantification. Baseline pNF- H values in NURTURE infants were obtained on Study Visit Day 1, either prior to nusinersen administration or four hours post-dose. See FIG. 6A of De Vivo (2019) Neuromuscul. Disord. 29(11):842-856. The neonatal subject levels are similar to typically developing infants and 35% of the lowest value in NURTURE study participants pre-risdiplam of FIG. 5. The data point shown in FIG. 4 at the pNF-H of 293 pg/mL correlates to the data point shown in FIG. 5.

The neurofilament levels in this Example can also be compared to limited published data on individuals with Type 1 SMA, untreated or treated with nusinersen or onasemnogene abeparvovec, and from typically developing infants. See Alves (2021) Mol. Ther. Methods Clin. Dev. 23:524-538; De Vivo (2019) Neuromuscl. Discord. 29:842-856; Darras (2019) Ann. Clin. Transl. Neurol. 6:932-944; and Pironkova (2017) Exp. Ther. Med. 14:228-238. Table 8 presents neurofilament levels in plasma (mean, pg/mL) reported in the literature.

TABLE 8

| | Plasma neurofilament | |
|---|---|---|
| | level (mean or median, | Plasma neurofilament |
| | range pg/mL) | level (mean or median, |
| | phosphorylated heavy | range pg/mL) |
| Neonatal population | chain (pNF-H) | light chain (NF-L) |
| Cord blood d0 (congenital | 137 ± 54 | |
| heart disease, CHD)¹ | | |
| Typically developing, <1 Y² | 1510 (579-7030) | |
| Typically developing, | 926 (177-7950) | 10 (3-19) |
| 57 d ± 102³ | | |
| “Pre-symptomatic,” <6 W, | 20881 (845-52900) | |
| 2 copies of SMN2⁴ | | |

See ¹Pironkova (2017) Exp. Th. Med. 14: 228-238; ²Darras (2019) ACTN 6: 932-44; ³Alves (2021) Molec.
Ther.: Methods and Clin. Dev. 524-38; and ⁴De Vivo (2019) Neuromusc. Dis. 29: 842-856.

Results from this Example suggest that the pNF-H levels are more informative than the lower and less responsive NF-L levels. Cord blood pNF-H levels reported in term neonatal subjects (mean 137±54 pg/ml, with a different assay) of the NURTURE study were approximately half that of the neonatal subject of this Example, suggesting a higher level of active axonopathy in the index case. See Pironkova (2017) Exp. Ther. Med. 14:228-238. By comparison “pre-symptomatic” neonatal subjects in the NURTURE study, under 6 weeks of age and with SMN2X2 had median (range) values of 20881 (845-52,900), e.g., this Example was 1.4% that of the median value, suggesting that the fetal subject exposure to risdiplam may have had a favorable response on stabilizing axonal pathology prenatally. See De Vivo (2019) Neuromuscl. Discord. 29:842-856.

In accordance with this Example, treatment of the carrier with oral risdiplam was safe and well tolerated. No treatment-related adverse events were identified by investigators or treating physicians in the carrier or the subject as a fetal subject or a neonatal subject. Risdiplam crosses the placenta from the carrier to the fetal subject achieving a therapeutically effective steady-state drug level in the fetal subject that was about two-thirds of the carrier level. A favorable pharmacodynamic effect was demonstrated with a higher SMN protein level and a lower neurofilament level in the neonatal subject at birth than in symptomatic infants or after initiation of oral treatment postnatally, suggesting that even a modest exposure to drug in utero may generate an increase in SMN protein that could be clinically meaningful. Clinical observations were favorable, including normal exams, high CHOP INTEND scores, and robust CMAP and MUNE.

### Example 2. A Phase H Open-Label, Single Arm, Multicenter Study to Evaluate the Safety, Pharmacokinetics and Efficacy of Risdiplam Administered to the Carrier for the Treatment

### of a Genetically Diagnosed SMA Fetal Subject

This is an open-label, single arm, multicenter, Phase II study to evaluate the safety, pharmacokinetics, pharmacodynamics, and efficacy of risdiplam administered to the carrier in the third trimester of pregnancy for the treatment of a fetal subject genetically diagnosed with SMA. As set forth in Table 9, the primary objectives of this study are to determine the safety of risdiplam in subjects with SMA treated in utero and their carriers following oral administration of risdiplam to the carrier during the third trimester of pregnancy and to evaluate the pharmacokinetics of risdiplam in carriers, fetal subjects, and neonatal subjects with SMA and assess pharmacodynamics (SMN protein) in subjects with SMA. The secondary objectives of this study are to evaluate efficacy of risdiplam in subjects with SMA on SMA biomarkers, achievement of motor function, survival and permanent ventilation and ability to feed.

TABLE 9

| | Corresponding Endpoint |
|---|---|
| Primary Objective(s) | |
| To evaluate the safety of risdiplam in | Number of carriers and/or subjects with SMA |
| the carrier, the fetal subject, and the | with adverse events and serious adverse events |
| neonatal subject | Number of carriers and/or subjects with SMA |
| | with related adverse events |
| | Number of carriers and/or subjects with SMA |
| | with adverse events by severity |
| | Number of carriers and/or subjects with SMA |
| | with adverse events leading to discontinuation |
| | Number of carriers and/or subjects with SMA |
| | with abnormal vital signs, ECG parameters, and |
| | clinical laboratory results |
| | Number of carriers with abnormal pregnancy |
| | outcomes (e.g., spontaneous abortions, stillbirths, |
| | elective abortions, pre-term births, post-term |
| | births) and pregnancy complications (e.g., |
| | preeclampsia, gestational diabetes) |
| | Number of subjects with SMA with major or |
| | minor congenital disorders |
| | Number of subjects with SMA at or below the |
| | third percentile of normal range for |
| | weight-for-age, length/height-for-age, and |
| | weight-for-length/height of age/visit based on |
| | the WHO Child Growth Standards (WHO 2019) |
| | Number of subjects with SMA at or below the |
| | third percentile of normal range for head |
| | circumference-for-age of age/visit based on the |
| | WHO Child Growth Standards (WHO 2019) |
| | Number of subjects with SMA with a scaled score |
| | below 1.5 standard deviation of chronological |
| | reference standard as measured by the Bayley |
| | Scales of Infant and Toddler Development, Third |
| | Edition (BSID-III) Cognitive Scale |
| | Number of subjects with SMA with abnormal |
| | findings in the neurological examination |
| To evaluate the PK of risdiplam in | PK: Plasma concentration of risdiplam (and its |
| carriers, fetal subjects, and neonatal | metabolite M1) at specified timepoints during |
| subjects with SMA and assess PD | pregnancy (carrier) and up to 8 weeks after birth |
| (SMN protein) in subjects with SMA | (neonatal subject) |
| | PD: SMN protein in neonatal subjects with SMA |
| | up to 8 weeks after birth |
| Secondary Objectives: Efficacy of | |
| Risdiplam in Subjects with SMA | |
| On SMA biomarkers | Compound muscle action potential (CMAP) |
| | amplitude at Week 2 and Months 1 and 2 for the |
| | neonatal subject |
| | Measurements of pNfH at birth and Months 1 and |
| | 2 for the neonatal subject |
| On the achievement of motor function | Total score in Children's Hospital of Philadelphia |
| | Infant Test of Neuromuscular Disorders |
| | (CHOP-INTEND) motor function scale for the |
| | neonatal subject at Month 2 |
| | Number of neonatal subjects who achieve a score |
| | of 40 or higher in the CHOP-INTEND motor |
| | function scale at Month 2 |
| On survival and permanent ventilation | Number of neonatal subjects alive without |
| | permanent ventilation at Month 2 |
| | Number of neonatal subjects alive at Month 2 |
| | Time to event (death, permanent ventilation, |
| | death, or permanent ventilation) up to Month 2 |
| | for the neonatal subject |
| On ability to feed | Number of neonatal subjects with the ability to |
| | swallow and to feed orally at Month 2 (as |
| | measured by Oral and swallowing abilities tool, |
| | or OrSAT) |

The study will enroll carriers aged 18 to 40 years who will start oral risdiplam between Weeks 28 and 30 of gestation after confirmation that all the eligibility criteria are met. The population of this study are carriers in the third trimester of pregnancy carrying a fetal subject with confirmed genetic diagnosis of SMA having homozygous deletion or heterozygosity predictive of loss of function of the SMN1 and 2 copies of SMN2 genes.

### Inclusion Criteria

[1]Potential subjects are eligible to be included in the study if all of the following criteria apply:
Carrier is a pregnant female aged>18 and <40 years
The carrier is overtly healthy and the fetal subject has normal development, as determined by the treating obstetrician's medical evaluation conducted according to locally applicable pregnancy care guidelines
The carrier has undergone (prior to enrollment) antenatal follow-up according to local guidelines and as determined by the treating obstetrician, which may include medical/obstetric/family history, current medicines history, nutrition, smoking and drug use history, mental health concerns, screening for infectious diseases, rhesus D status, screening for fetal anomalies, and other risk factors assessment (e.g., gestational diabetes, pre-eclampsia, venous thromboembolism)
The carrier has a body mass index<32 kg/m2 before pregnancy and weight gain during gestation within recommended limits based on pre-pregnancy weight as determined by the treating obstetrician
Fetal subject with a confirmed prenatal diagnosis of SMA as previously determined before screening (and documented in the carrier's medical history) through local laboratory testing by amniocentesis or chorionic villus sampling cells having homozygous deletion or heterozygosity predictive of loss of function of the SMN1 gene and 2 copies of SMN2 gene

### Exclusion Criteria

[2]Potential subjects are excluded from the study if any of the following criteria apply:
Fetal subjects with major congenital malformations or other fetal anomalies considered to be clinically significant by the treating obstetrician
Multiple gestation (twins or other multiples)
Conditions considered as factors for high-risk pregnancy, such as hypertension, diabetes, placental abnormalities, blood clotting disorders, or other problems which in the opinion of the treating obstetrician pose a serious concern to the outcome of the ongoing pregnancy
Exposure to genotoxic substances including medications and/or ambient/environment factors within 6 months plus 5 elimination half-lives since last exposure prior to or during pregnancy
Treatment with any non-genotoxic teratogenic medication within 1 month or 5 elimination half-lives (whichever is longer) prior to or during pregnancy
Treatment with investigational therapy within 1 month or 5 elimination half-lives whichever is longer prior to or during pregnancy
The carrier taking any of the following:
   Any inhibitor of CYP3A4 taken within 2 weeks (or within 5 times the elimination half-life, whichever is longer) prior to first dose of risdiplam, including but not limited to: ketoconazole, miconazole, itraconazole, fluconazole, erythromycin, clarithromycin, ranitidine, cimetidine, and including food or drinks known to modulate CYP3A activity (e.g., Seville oranges, grapefruit or grapefruit juice, pomelos, exotic citrus fruits, grapefruit hybrids, or fruit juices)
   Any inducer of CYP3A4 taken within 4 weeks (or within 5 times the elimination half-life, whichever is longer) prior to first dose of risdiplam, including but not limited to: rifampicin, rifabutin, glucocorticoids, carbamazepine, phenytoin, phenobarbital, or St. John's wort
   Any multidrug and toxin extrusion (MATE) substrates taken within 2 weeks (or within 5 times the elimination half-life, whichever is longer) prior to first dose of risdiplam
Any serious medical condition or abnormality in clinical laboratory tests that precludes safe participation in and completion of the study

The carrier will receive risdiplam from Week 28-30 of gestation until delivery of the fetal subject as the neonatal subject. Gestational age should be estimated by the most accurate method as determined by the treating obstetrician. Risdiplam will be administered orally at a dose of 5 mg once daily. The neonatal subject will receive risdiplam after birth orally once daily at a dose of 0.15 mg/kg body weight for the first two months from birth and at a dose of 0.2 mg/kg body weight after two months until two years of age.

Risdiplam treatment will start at an oral dose of 5 mg once daily for the carrier. Blood samples will be obtained from the carrier during pregnancy to assess the risdiplam levels. If the carrier was to require an amniocentesis during the third trimester for clinical reasons, samples of amniotic fluid and fetal cord blood may be collected to monitor risdiplam exposure of the fetal subject. Treatment may be stopped if any of the stopping rules are met.

Safety monitoring of the fetal subject will consist of standard of care (SOC) for the level of pregnancy risk and include as a minimum a biophysical profile and full anatomical and growth ultrasound. Assessment of carrier safety will include collection of adverse events or serious adverse events, including selected abnormal pregnancy outcomes (e.g., spontaneous abortions, stillbirths, elective abortions, pre-term births, and post-term birth) and pregnancy complications. Assessment of the safety of risdiplam in the subjects with SMA treated in utero will include collection of adverse events or serious adverse events (including major and minor congenital disorders, small for gestational age, pre- and postnatal growth, and cognitive and neurological development) up to the first two years of life of the neonatal subject.

Once the fetal subject is delivered as the neonatal subject, the carrier will stop treatment with risdiplam. At the time of delivery, samples of umbilical cord blood (venous and arterial) and amniotic fluid will be collected, if possible, to assess the in utero exposure of the fetal subject to risdiplam.

The day after birth, the neonatal subject will start oral treatment with risdiplam.

Blood samples will be obtained from the neonatal subject to assess risdiplam levels and SMN protein.

After delivery, monitoring of the neonatal subject will include study assessments, which include a brain ultrasound scan, safety labs (hematology, chemistry panel), vital signs, and ECG. Additionally, the child will undergo the following assessments: Module 2 of the Hammersmith Infant Neurologic Examination (HINE-2), Children's Hospital of Philadelphia Infant Test of Neuromuscular Disorders (CHOP-INTEND), Bayley Scales of Infant and Toddler Development-Third Edition (BSID-III), oral and swallowing abilities tool (OrSAT), compound muscle action potential (CMAP) amplitude, and phosphorylated neurofilament heavy chain (pNfH).

A neonatal subject is considered small for its gestational age if it is at or below the third percentile of normal range for weight-for-age, length/height-for-age, and weight-for-length/height of age/visit and head circumference-for-age of age/visit based on the World Health Organization (WHO) Child Growth Standards. A neonatal subject is considered to have a cognitive deficit if it has a scaled score below 1.5 standard deviation of chronological reference standard as measured by the Bayley Scales of Infant and Toddler Development, Third Edition (BSID-III) Cognitive Scale.

Pharmacokinetic and pharmacodynamic evaluation of both the carrier and the neonatal subject may include measurement of plasma concentration of risdiplam (and its metabolite M1) at specified timepoints during pregnancy (carrier) and up to 8 weeks after birth (neonatal subject) and measurement of SMN protein in neonatal subjects with SMA up to 8 weeks after birth.

The evaluation of efficacy of risdiplam in neonatal subjects treated in utero will be based on SMA biomarkers as early predictors of benefit (CMAP amplitude and neurofilament) and clinical outcomes assessing motor function and milestones. These outcomes have been extensively used in previous SMA clinical trials and can be objectively assessed. The results of efficacy assessments will be contextualized with corresponding data in subjects treated presymptomatically, as well as normative data in healthy neonatal subjects, where available.

The neonatal subject will continue to be observed for safety and efficacy for two years, and the carrier will be observed for safety until two months after delivery of the fetal subject as the neonatal subject. FIG. 6 is a schematic diagram of a study schema associated with Example 2. The primary analysis will be performed once the last neonatal subject in the study completes the Month 2 study visit.

### OTHER EMBODIMENTS

This application refers to various published patent applications, journal articles, and other publications, each of which is incorporated herein by reference.

The foregoing has been described of certain non-limiting embodiments of the present disclosure. Those of ordinary skill in the art will appreciate that various changes and modifications to this description may be made without departing from the spirit or scope of the present disclosure, as defined in the following claims.

1-74. (canceled)

75. A method of treating spinal muscular atrophy (SMA) in a fetal subject in need thereof, the method comprising administering risdiplam to the carrier of the fetal subject.

76. The method of claim 75, wherein the fetal subject is in its third trimester of development.

77. The method of claim 75, wherein the fetal subject has two or less survival motor neuron 2 (SMN2) gene copies.

78. The method of claim 75, wherein the fetal subject has two survival motor neuron 2 (SMN2) gene copies.

79. The method of claim 75, wherein the risdiplam is orally administered daily to the carrier of the fetal subject.

80. The method of claim 75, wherein 5 mg of risdiplam is orally administered daily to the carrier of the fetal subject.

81. The method of claim 75, wherein:
the fetal subject is in its third trimester of development; and
5 mg of risdiplam is orally administered daily to the carrier of the fetal subject.

82. The method of claim 81, wherein the fetal subject has two copies of the survival motor neuron 2 (SMN2) gene.

83. The method of claim 81, wherein the method further comprises terminating administration of risdiplam to the carrier of the fetal subject upon delivery of the fetal subject from the carrier as a neonatal subject.

84. A method of treating spinal muscular atrophy (SMA) in a neonatal subject in need thereof, the method comprising administering to the neonatal subject a therapeutically effective amount of risdiplam, wherein when the neonatal subject was a fetal subject the fetal subject received risdiplam in utero through the carrier of the fetal subject.

85. The method of claim 84, wherein the neonatal subject is aged from birth to two months, and 0.15 mg/kg body weight of risdiplam is orally administered daily to the neonatal subject.

86. The method of claim 84, wherein the neonatal subject is between two months and two years of age, and 0.20 mg/kg body weight of risdiplam is orally administered daily to the neonatal subject.

87. The method of claim 84, wherein the neonatal subject has two copies of the survival motor neuron 2 (SMN2) gene.

88. The method of claim 84, wherein the neonatal subject has Type 1 SMA.

89. The method of claim 84, wherein the neonatal subject has Type 0 SMA.

90. A method of treating spinal muscular atrophy (SMA) in a subject in need thereof, the method comprising:
administering risdiplam to a carrier of a fetal subject in a carrier dosing cycle to administer to the fetal subject a therapeutically effective amount of risdiplam, wherein the fetal subject has SMA;
terminating the carrier dosing cycle when the fetal subject is delivered from the carrier as a neonatal subject; and
administrating an SMA therapy directly to the neonatal subject.

91. The method of claim 90, wherein administration of risdiplam to the carrier is initiated when the fetal subject is at least twenty-eight weeks gestation.

92. The method of claim 90, wherein 5 mg of risdiplam is orally administered daily to the carrier of the fetal subject.

93. The method of claim 90, wherein:
the SMA therapy administered directly to the neonatal subject is risdiplam;
the neonatal subject is aged from birth to two months; and
0.15 mg/kg body weight of risdiplam is orally administered daily to the neonatal subject.

94. The method of claim 93, wherein administration of risdiplam to the neonatal is initiated when the level of risdiplam in blood of the neonatal subject is less than 100 ng/mL.

95. The method of claim 94, wherein the level of risdiplam in blood of the neonatal subject is measured in the cord blood of the neonatal subject at the time of delivery.

96. The method of claim 90, wherein:
the SMA therapy administered directly to the neonatal subject is risdiplam,
the neonatal subject is between two months and two years of age; and
0.20 mg/kg body weight of risdiplam is orally administered daily to the neonatal subject.

97. The method of claim 90, wherein the fetal subject has two copies of the survival motor neuron 2 (SMN2) gene.

98. The method of claim 90, wherein the neonatal subject has Type 1 SMA.

99. The method of claim 90, wherein the neonatal subject has Type 0 SMA.

100. A method of treating spinal muscular atrophy (SMA) in a fetal subject in need thereof, the method comprising administering to the fetal subject a therapeutically effective amount of risdiplam, wherein the fetal subject receives the risdiplam in utero through a carrier of the fetal subject.

101. The method of claim 100, wherein the fetal subject is at least twenty-eight weeks gestation.
